(19) United States
(12) Patent Application Publication
(10) Pub. No.: US 2020/0187863 A1
(43) Pub. Date: Jun. 18, 2020

(54) Sterile Urine Collection Mechanism for Medical Diagnostic Systems

(71) Applicant: Voyant Diagnostics, Inc., Chicago, IL (US)

(72) Inventors: Michael Tu, Skokie, IL (US); Claire Zhou, Chicago, IL (US); Peter Nebres, Chicago, IL (US); Prasanth Bijjam, Schaumburg, IL (US); Eric Shain, Glencoe, IL (US); Craig Sampson, Lake Bluff, IL (US)

(21) Appl. No.: 16/712,748

(22) Filed: Dec. 12, 2019

Related U.S. Application Data
(63) Continuation-in-part of application No. 16/015,417, filed on Jun. 22, 2018.
(60) Provisional application No. 62/524,199, filed on Jun. 23, 2017.
(60) Provisional application No. 62/779,560, filed on Dec. 14, 2018.
(60) Provisional application No. 62/802,768, filed on Feb. 8, 2019.
(60) Provisional application No. 62/823,939, filed on Mar. 26, 2019.
(60) Provisional application No. 62/848,107, filed on May 15, 2019.
(60) Provisional application No. 62/866,067, filed on Jun. 25, 2019.
(60) Provisional application No. 62/937,852, filed on Nov. 20, 2019.

(51) Int. Cl. A61B 5/00; A61B 10/00; A61B 5/20; A61B 90/96; A61B 5/145; E03D 11/13

(52) U.S. Cl. CPC A61B 5/6891; A61B 10/007; A61B 5/207; A61B 90/96; A61B 90/361; A61B 5/4875; A61B 10/0012; E03D 11/13; A61B 2562/0295; A61B 5/14546

(57) ABSTRACT

Example embodiments relate to a sterile urine collection mechanism for medical diagnostic systems. An example device includes an initial collection component configured to collect a midstream urine sample from a patient. The device also includes a plurality of test strips configured to indicate a condition of the patient at a point of care. In addition, the device includes a sensor configured to capture an image of at least one test strip exposed to a portion of the midstream urine sample. Further, the device includes a computing device configured to analyze the image of the test strip captured by the sensor in order to determine the condition of the patient. Even further, the device includes a motor configured to position the test strip near the sensor. Yet further, the device includes an additional collection component configured to collect an additional portion of the midstream urine sample for central laboratory testing.

### CROSS-REFERENCE TO RELATED APPLICATIONS

The present application is a Continuation-in-Part application claiming priority to U.S. patent application Ser. No. 16/015,417, filed Jun. 22, 2018, which itself claims the benefit of priority of U.S. Patent Application No. 62/524,199, filed Jun. 23, 2017. The present application also claims the benefit of priority of U.S. Patent Application No. 62/779,560, filed Dec. 14, 2018; U.S. Patent Application No. 62/802,768, filed Feb. 8, 2019; U.S. Patent Application No. 62/823,939, filed Mar. 26, 2019; U.S. Patent Application No. 62/848,107, filed May 15, 2019; U.S. Patent Application No. 62/866,067, filed Jun. 25, 2019; and U.S. Patent Application No. 62/937,852, filed Nov. 20, 2019.

The present application hereby incorporates by reference, in their entireties, U.S. Patent Application No. 62/524,199, filed Jun. 23, 2017; U.S. patent application Ser. No. 16/015,417, filed Jun. 22, 2018; U.S. Patent Application No. 62/779,560, filed Dec. 14, 2018; U.S. Patent Application No. 62/802,768, filed Feb. 8, 2019; U.S. Patent Application No. 62/823,939, filed Mar. 26, 2019; U.S. Patent Application No. 62/848,107, filed May 15, 2019; U.S. Patent Application No. 62/866,067, filed Jun. 25, 2019; and U.S. Patent Application No. 62/937,852, filed Nov. 20, 2019.

### FIELD OF THE INVENTION

The present disclosure relates to a sterile urine collection mechanism as part of a medical diagnostic system.

### BACKGROUND

Unless otherwise indicated herein, the materials described in this section are not prior art to the claims in this application and are not admitted to be prior art by inclusion in this section.

Currently there exists different methods to testing urine. However, to automate testing and bring the lab testing closer to the patient, the sample could be collected in the toilet. If the toilet is used by multiple consecutive patients, it is difficult to, as of now, capture sterile urine. Hence, a conventional alternative of collecting urine samples using a cup may be used instead.

Women of child-bearing age visit U.S. emergency departments (ED) an estimated 33.6 million times each year. Clinical standards recommend administering a point-of-care pregnancy test for this population since exclusion of pregnancy based on menstrual history is often not reliable on its own. The most common point-of-care test performed is a human chorionic gonadotropin (hCG) urine test. This intervention ensures that no woman of child-bearing age in the ED is put at risk while the provider is weighing diagnostic and/or treatment options. This clinical step is also important in other acute care settings (e.g., urgent care centers).

Minimizing risk of potential harm towards a fetus, especially in the most sensitive first trimester, is critical. Treatment plans that include radiological testing, anesthetic procedures, and prescription of teratogenic (category D or X) drugs all come with fetal risks. Such exposure can lead to growth retardation, congenital malformation, impaired brain function, childhood cancer, and miscarriage. In addition to adverse patient outcomes, pregnancy misdiagnosis can lead to repeat ED visits and medicolegal costs.

Current point-of-care pregnancy testing in the acute care setting is inadequate for at least two reasons: 1) in practice, implementation of the screening guideline is low, and 2) when the test is administered, user errors on the part of clinical staff can lead to unreliable results. Pregnancy testing in the ED is a time consuming, laborious, and complex process than can take up to 65 minutes. The standard, point-of-care urine test used today is not well-suited for high throughput, rapid mass screening.

Only an estimated 27% of acute care visits by women of child-bearing age include pregnancy testing. This lack of compliance persists even in situations where risky clinical action is taken. For example, a majority of ED visits by reproductive-aged women in which patients are prescribed teratogenic medications do not include a pregnancy test. This insufficient 27% of incoming women estimated to be screened leaves potentially 25 million unscreened women in emergency departments. Furthermore, an estimated 10% of women of child-bearing age are typically pregnant. With consideration of all these statistics, up to 2.5 million pregnant women are put at risk each year—up to half of all pregnancies.

Point-of-care urine tests, including pregnancy tests, are essentially waived from oversight. However, waived tests are often done incorrectly. The non-laboratory staff typically involved in point-of-care testing are often inadequately trained. User errors can include misplacement of samples, mislabeling of samples, testing process error, inaccurate visual interpretation, and incorrect entry of results into the electronic health record (EHR) system. Government spot checks of facilities that conduct point-of-care tests have found less than 50% compliance with policies meant to ensure proper care.

Of those women that are screened, the point-of-care pregnancy tests used in the emergency department are typically the same or similar tests as used at home. Such home tests are not designed for high throughput, rapid mass screening, or other needs of a typically busy emergency department. Current testing practices may also increase user errors that lead to false negatives, including misplacement of samples, mislabeling of samples, testing process errors, inaccurate visual interpretation, and incorrect entry of results into an electronic health record.

Of those women that are screened, they have to urinate into a cup to collect the sample. Urinating into a cup can be difficult for healthy patients, and may require assistance from medical staff for patients who are older, disabled, or too sick to do so by themselves. Either the patient or the medical staff will then need to carry the urine to a counter or to the lab for analysis, creating dissatisfaction for both the patient and staff

Furthermore, it is estimated that nearly one hundred thirty million people are screened annually for routine urinalysis tests across multiple ambulatory settings, including, but not limited to, the emergency room, urgent care clinics, and private offices such as obstetrician/gynecologist offices and urologist offices.

Routine urinalysis exams consist of three tests: visual, chemical, and microscopic. Typically, visual tests and chemical tests are performed at the point-of-care. With visual tests, the urine's appearance is examined for turbidity and color. With chemical tests, currently, the urine is analyzed using a dipstick test with chemical strips that change colors if certain substances are present or if their levels are above normal. The clinical standard is a 10-panel assay that includes glucose, bilirubin, ketone, specific gravity, blood, pH, protein, urobilinogen, nitrite, and leukocyte esterase.

Furthermore, urine drug testing is performed to screen for the presence of certain illegal drugs and prescription drugs including amphetamines, methamphetamines, benziodiazepines, barbiturates, marijuana, cocaine, phencyclidine (PCP), methadone, and opioids. Drug testing can be performed by the primary care physician to test for possible substance abuse. Employers can require employees to perform drug tests prior to being hired or during the course of their employment, in particular if the employees are required to be alert during the job. Drug and alcohol rehabilitation centers can perform drug tests on their patients in order to determine whether they are continuing to use drugs and/or alcohol. Drug testing can also be performed in the home setting to see if family members are using drugs.

Furthermore, it is estimated that more than two hundred million people are undiagnosed for chronic kidney disease (CKD) globally. Chronic kidney disease is largely undiagnosed because it is asymptomatic, so regular testing may be overlooked. Chronic kidney disease progresses to kidney failure. Medicare is estimated to spend tens of billions of dollars per year to treat kidney failure. This number has been estimated to scale to over a half-trillion dollars of spending per year to treat kidney failure globally if most cases were treated. Unfortunately, many developing countries cannot treat kidney failure because the cost is prohibitive. Hence, diagnosing early-stage chronic kidney disease is paramount.

To cost effectively diagnose previously undiagnosed chronic kidney disease patients, The American College of Physicians recommends screening at-risk people with hypertension (estimated 1 billion people globally), people with diabetes (estimated 422 million people globally), and people above the age of 60 for chronic kidney disease. Furthermore, The American Society of Nephrology strongly recommends routinely screening all adults for chronic kidney disease to diagnose chronic kidney disease in its early stages when its progression can be halted.

Known as the “Silent Killer,” chronic kidney disease is asymptomatic in its early stages, leaving an estimated 10 million adults in the U.S. undiagnosed. While only a small percentage of patients advance to end-stage kidney failure, treatment for those who do is costly. Nearly 6% of Medicare expenditures come from the 1% of covered patients who have end-stage renal failure. Including the cost to other payors and out-of-pocket expenses, the total annual bill for treating kidney failure is estimated at over $35 billion.

Furthermore, chronic kidney disease is highly co-morbid with other fatal chronic diseases. Beyond reducing the financial burden of end-stage kidney failure, managing chronic kidney disease early can also reduce the mortality rate and costs related to cardiovascular disease and diabetes among a much larger patient population. The American Society of Nephrology strongly recommends routinely screening all adults for chronic kidney disease to diagnose the disease in its early stages when its progression can be halted.

Screening and/or monitoring for biomarkers that indicate chronic kidney disease such as albumin to creatinine ratio or the level of beta-trace protein at the point of care is critical for chronic kidney disease. Point-of-care testing (PoCT) could improve adherence to screening recommendations and patient outcomes. Point-of-care testing is known to have a positive impact on operational efficiency and patient care. Such devices bring testing closer to the patient and provide physicians with faster results to expedite diagnosis and subsequent treatment. However, there are barriers to adoption. Physicians are often concerned about the reliability of test results from point-of-care testing. Errors can occur in the analytic phase of testing due to human error on the part of non-laboratory staff who are typically involved in current point-of-care testing techniques.

Therefore, a need exists to solve the deficiencies present in the prior art. What is needed is a system to facilitate testing of samples for biomarkers indicative of a medical condition. What is needed is a system to facilitate collection of urine for substantially automated testing. What is needed is a system to automate testing of samples using optically and/or electronically detectable indicators. What is needed is a system to communicate detected biomarkers indicative of a condition to a network-connected electronic computing device. What is needed is a method of substantially automated collecting, processing, testing, and optically and/or electronically analyzing indicators to predict a medical condition. What is needed is a method including a substantially automated test for and detection of indicators of chronic kidney disease, pregnancy, and/or other medical conditions within an acceptable margin of error.

### SUMMARY

The specification and drawings disclose embodiments that relate to a sterile urine collection mechanism as part of a medical diagnostic system. Embodiments disclosed herein will allow for patients to urinate normally while collecting sterile samples for both the automated diagnostic process attached to the toilet and potential lab testing in the future.

The sample collection mechanism described in this disclosure captures the ability to get sterile samples, a modular platform that may be placed on any toilet, the ability to perform midstream catch of urine (or “clean catch”), the ability to transfer urine from the sterile mechanism to a sterile collection cup for further lab testing, the ability to transfer urine from a sterile mechanism to a separate diagnostic device, the ability to transfer the sterile container to a different place, and the ability to clean and maintain the platform.

In a first aspect, the disclosure describes a device. The device includes an initial collection component configured to collect a midstream urine sample from a patient. The device also includes a plurality of test strips configured to indicate a condition of the patient when exposed to the midstream urine sample. Further, the device includes a fluid transportation system. The fluid transportation system is configured to transport a portion of the midstream urine sample from the initial collection component to at least one test strip of the plurality of test strips. The fluid transportation system is also configured to expose the at least one test strip to the portion of the midstream urine sample. In addition, the device includes a sensor configured to capture an image of the at least one test strip exposed to the portion of the midstream urine sample. The image of the at least one test strip indicates the condition of the patient at a point of care. Still further, the device includes a computing device configured to analyze the image of the at least one test strip captured by the sensor in order to determine the condition of the patient at the point of care. Even further, the device includes a motor configured to position the at least one test strip near the sensor after the at least one test strip is exposed to the portion of the midstream urine sample. Yet further, the device includes an additional collection component configured to collect an additional portion of the midstream urine sample from the initial collection component for central laboratory testing.

The device may include the following optional features. The device may include a midstream urine collection seat. The midstream urine collection seat may include the initial collection component. The seat may be raised or lowered mechanically using an electric motor. The seat may be mechanically lowered into a lowered position to collect urine from the patient for testing. The seat may be mechanically raised into a raised position after urine has been collected from the patient. The additional collection component may include a sterile collection cup. The sterile collection cup may have one or more barcode stickers disposed thereon. A barcode on the one or more barcode stickers may match a barcode on a bracelet worn by the patient. The initial collection component may include a rigid central platform with a film overlaid over the platform to absorb an initial urine stream and collect the midstream urine sample. The rigid central platform may include an anti-microbial coating and a hydrophobic coating, the film may include a trough in a center of the film, the film may include absorbent strips spaced at discrete intervals on the film to absorb the initial urine stream and collect the midstream urine sample (the absorbent strips including sodium polyacrylate), the film may include holes, or the film may include shapes made of water-soluble plastic. The film may be stabilized using a guide wire. The film may include two circular layers of water-soluble, polyvinyl alcohol (PVA) over which the midstream urine sample collects. The midstream urine sample may dissolve the two circular layers within a few seconds of collecting over the two circular layers. The initial collection component may include a rigid central platform made of polyoxymethylene plastic that is coated with an anti-microbial coating and a hydrophobic coating. The initial collection component may include a rigid central platform that includes an array of holes connected to a membrane and a vacuum pump located inside the rigid central platform. The initial collection component may include a film and a rigid contoured base. The initial collection component may include a backside curvature configured to hold the midstream urine sample when the initial collection component is raised. The initial collection component may include a film. After use, the film may be unrolled from over the initial collection component and a replacement film may be rolled over the initial collection component. The device may include an instruction screen configured to provide a demonstration on how to use the device. The demonstration may include static drawings, videos, or audio. The initial collection component may include a platform having an open space defined therein for disposal of toilet paper into a toilet bowl below the platform. The additional collection component may include a sterile collection cup. The device may also include a predefined location for the additional collection component. The predefined location may be revealed using an electronic signal or mechanical method. The initial collection component may include a rigid platform overlaid with film. The platform may be positioned over a toilet bowl. The additional collection component may include a sterile collection cup. The platform may be raised in response to a flush lever of the toilet being pushed. The midstream urine sample may be at least partially transferred from the collection component to the additional collection component upon the platform being raised. The device may also include a refillable reservoir configured to provide clean water for flushing and device sterilization functions. The device may also include a battery backup. The device may be mobile and portable. Transporting the portion of the midstream urine sample from the initial collection component to the at least one test strip of the plurality of test strips may be performed using a y-shaped tubing apparatus controlled by a three-way solenoid valve and a tubing pathway. The tubing pathway may bifurcate into a first tubing pathway and a second tubing pathway. The first tubing pathway may be directed to the at least one test strip. The second tubing pathway may be directed to a waste basin in the device and then to a toilet bowl. The at least one test strip may be configured to indicate the presence of a uremic toxin, a biomarker associated with cardiovascular disease, or a biomarker associated with chronic kidney disease. The uremic toxin, the biomarker associated with cardiovascular disease, or the biomarker associated with chronic kidney disease may include urea, phosphate, to creatinine ratio, creatinine, parathyroid hormone (PTH), beta 2 microglobulin, cystatin C, myoglobin, kappa free light chains, complement factor D, interleukin-6, alpha 1 microglobulin, YKL-40, lambda free light chains, albumin, indoxyl sulfate, indoxyl glucuronide, indoleacetic acid, P-Cresyl sulfate, P-Cresyl glucuronide, phenyl sulfate, phenyl glucuronide, phenylacetic acid, phenylacethyl glutamine, hippuric acid, 4-Ethylphenyl sulfate, or 3-Carboxy-4-methyl-5-propyl-2-furanpropionic acid. The device may also include an additional sensor configured to capture an image of the midstream urine sample. The image of the midstream urine sample may indicate an extent of dehydration of the patient. The computing device may be configured to analyze the image of the midstream urine sample in order to determine the extent of dehydration of the patient by determining a color and darkness of the midstream urine sample. The device may also include a refractometer. The at least one test strip may indicate the extent of dehydration of the patient. The computing device may be configured to analyze the image of the at least one test strip or data from the refractometer in order to determine the extent of dehydration of the patient by determining a concentration of the midstream urine sample or a specific gravity of the midstream urine sample. The condition of the patient may include ovulation. The plurality of test strips may be configured to indicate the presence of luteinizing hormone. The device may be configured to fit around or over consumer toilets in an at-home setting or a senior care setting.

In a second aspect, the disclosure describes a system. The device includes an initial collection component configured to collect a midstream urine sample from a patient. The device also includes a plurality of test strips configured to indicate a condition of the patient when exposed to the midstream urine sample. Further, the device includes a fluid transportation system. The fluid transportation system is configured to transport a portion of the midstream urine sample from the initial collection component to at least one test strip of the plurality of test strips. The fluid transportation system is also configured to expose the at least one test strip to the portion of the midstream urine sample. In addition, the device includes a sensor configured to capture an image of the at least one test strip exposed to the portion of the midstream urine sample. The image of the at least one test strip indicates the condition of the patient at a point of care. Still further, the device includes a computing device configured to analyze the image of the at least one test strip captured by the sensor in order to determine the condition of the patient at the point of care. Even further, the device includes a motor configured to position the at least one test strip near the sensor after the at least one test strip is exposed to the portion of the midstream urine sample. Yet further, the device includes an additional collection component configured to collect an additional portion of the midstream urine sample from the initial collection component for central laboratory testing. The system also includes a toilet bowl onto which the device is attached.

In a third aspect, the disclosure describes a method. The method includes collecting a midstream urine sample from a patient in an initial collection component. The method also includes transporting a portion of the midstream urine sample from the initial collection component to at least one test strip using a fluid transportation system. Further, the method includes exposing, by the fluid transportation system, the at least one test strip to the portion of the midstream urine sample. The at least one test strip is one of a plurality of test strips configured to indicate a condition of the patient when exposed to the urine sample. In addition, the method includes positioning, by a motor, the at least one test strip near a sensor. Even further, the method includes capturing an image of the at least one test strip using the sensor. The image indicates the condition of the patient at a point of care. Yet further, the method includes analyzing, by a computing device, the image of the at least one test strip in order to determine the condition of the patient at the point of care. Still further, the method includes collecting, from the initial collection component, an additional portion of the midstream urine sample in an additional collection component for central laboratory testing.

Another aspect of the disclosure provides a system to facilitate testing of samples for biomarkers indicative of a medical condition. An aspect of the disclosure provides a system to facilitate collection of urine for substantially automated testing. An aspect of the disclosure provides a system to automate testing of samples using optically and/or electronically detectable indicators. An aspect of the disclosure provides a system to communicate detected biomarkers indicative of a condition to a network-connected electronic computing device. An aspect of the disclosure provides a method of substantially automated collecting, processing, testing, and optically and/or electronically analyzing indicators to predict a medical condition. An aspect of the disclosure provides a method including a substantially automated test for and detection of indicators of chronic kidney disease, pregnancy, and/or other medical conditions within an acceptable margin of error.

Another aspect of the disclosure allows for the determination of dehydration and if dehydration exists, the extent of dehydration of the patient by measuring the color and darkness of the urine. In one embodiment, an image of the urine may be taken by any suitable means such as a camera and the image processed through an image processing algorithm. In another embodiment, the device and system may measure the dehydration of the patient by measuring the concentration and/or the specific gravity of the urine with a general urinalysis test strip and/or a refractometer. In another embodiment, the device and system may measure dehydration by monitoring the frequency of urination of the patient. For example, the frequency of urination may be measured based on the number of times the device is used or the number of times the toilet is flushed per unit time. The frequency of urination may also be measured by the number of times the wristband is scanned. In other embodiments, the frequency of urination may be measured through patient self-reporting via a mobile or web application.

Another aspect of the disclosure allows weight and/or force sensors and/or scales to be embedded into or onto the toilet seat component of the sample collection mechanism to measure the weight of the patient when they sit on the urine collection subsystem.

Another aspect of the disclosure may be used to perform an ovulation test on a person's urine by detecting luteinizing hormone in the urine using test strips that target luteinizing hormone. Test strips that target luteinizing hormone may be incorporated into test strip cartridges as described herein to detect luteinizing hormone in the urine. The test strips may be antibody-based lateral flow strips. Example devices may perform a one-time test per person or the device may continuously monitor a person for luteinizing hormone continuously over time. Additionally, example devices may be deployed in a clinical setting or in the home to perform the ovulation test. The luteinizing hormone test may be qualitative, semi-quantitative, or quantitative (e.g., the luteinizing hormone test may result in a concentration of luteinizing hormone). In some embodiments, a concentration of luteinizing hormone may be compared to a threshold concentration to make a determination about ovulation. The threshold concentration may be based on one or more characteristics of the patient (e.g., height, weight, age, etc.).The test strips used to target luteinizing hormone may include antibody-based lateral flow urine test strips. Such test strips may be selected by the medical staff or the patient using a mobile application or web application. Selections may also be automated by scanning the barcode on the patient's wristband. The image processing algorithm may be adapted to capture quantitative, semi-quantitative, or qualitative tests.

Another aspect of the disclosure may be miniaturized to fit around or over consumer toilets in the at-home setting or commercial toilets in a clinical setting. In particular, the urine analysis sub-system may be wrapped around the toilet tank or be fitted on top of the toilet tank or a combination of both. The urine analysis sub-system may also be fitted in between the toilet tank and toilet seat. When the urine analysis sub-system is wrapped around the toilet tank, the urine analysis sub-system may also be fitted in between the toilet tank and toilet seat. In some embodiments, the urine analysis subsystem may be made smaller. Further, the urine collection subsystem may be made smaller. Both the urine analysis subsystem and the urine collection subsystem may be adhered through mechanical fittings or clamps or connectors onto the toilet. In some embodiments, test strip cartridges may also be made smaller. In addition, multiple models, dimensions, and form factors are contemplated herein so as to fit over variously sized toilets and toilet seats. In some embodiments, the collection mechanism may be designed to have clamps that allow it to hook onto the bottom of a standard toilet seat.

Applications based on this disclosure may include substantially automated urine pregnancy testing, for example, in the emergency department through urine testing of human chorionic gonadotropin (hCG). This emergency department use case may automate away or substantially reduce user error. Previously, examples of user errors may occur when nurses fail to label the urine cups with patient identification. As a result, the urine sample for a patient can get switched up, which can lead to a false negative result. This problem can lead to females who are pregnant being treated in the emergency department or urgent care facility as if they are not pregnant, potentially exposing the fetus to drugs and radiation that can harm it for the rest of its life. The substantially automated testing of this disclosure may reduce the risk of this devastating problem.

Solutions provided throughout this disclosure are intended to automate the pregnancy testing process in the emergency department, providing results in typically less than 5 minutes without clinical and non-clinical staff input. In one example, the system may be installed on a toilet, with the device resting on the toilet tank. The device is connected to a urine collection cup that is installed on or inside the toilet bowl. The patient will typically be able to scan his or her hospital barcode ID on the device before urinating into the collection cup that is installed on or inside the toilet bowl. The add-on device to the toilet may automatically collects, process, and analyze the urine to determine the pregnancy status of the patient. The system then may automatically send the test results to an electronic health record.

Solutions provided throughout this disclosure substantially automate the entire emergency department pregnancy testing process from the point of specimen collection to sending the test result to the electronic health record. The system also substantially automates the sample processing, dispensing, testing, analysis, and cleaning processes. The system substantially automates the cleaning processes of the device, its collection cup, and the connections between the device and its collection cup. The system also substantially automates the test strip usage process.

The solutions to the deficiencies in the prior art provided throughout this disclosure are intended to improve patient outcomes without increasing the clinical burden. Eliminating labor costs and decreasing testing time, systems included by this disclosure may substantially increase, for example, double, the number of women screened typically without increasing overall cost. Removing user errors also drives more reliable results. The technology discussed throughout this disclosure is superior to existing manual and semi-automated urine pregnancy tests in both speed and reliability.

Various embodiments of the solution described throughout this disclosure facilitate previously undiagnosed chronic kidney disease (CKD) to be diagnosed through time-efficient, relatively inexpensive, and accurate mass screening. Various embodiments described throughout this disclosure may provide for an add-on device installable to toilets or other existing devices that substantially automatically analyzes urine for biomarkers of a detectable condition, for example, chronic kidney disease. Biomarkers may include, without limitation, beta-trace protein, albumin, and creatinine. Collectively, the biomarkers may provide substantially accurate indicators of a detectable condition, for example CKD or pregnancy, from its early through late stages. The solutions provided throughout this disclosure may be installed onto an existing device found at a testing location. For example, solutions described throughout this disclosure may be installed on a toilet in a physician's office, a clinic, such as a walk-in clinic, patient home, and/or a screening van as a point-of-care screening device for CKD and other conditions. Patients, for example, adults above the age of 45 or with a previous history of hypertension or diabetes, may visit a screening facility, simply urinate into a device of this disclosure, and quickly be diagnosed for a medical condition, for example CKD, in its early stages.

Solutions provided throughout this disclosure may enable emergency departments, urgent care facilities, and other locations to implement best practices, including mass pregnancy screening. This disclosure provides a solution to the longstanding problem that, despite clinical standards, only about 27% of emergency department visits by women of childbearing age include pregnancy testing because the existing urine testing process for pregnancy is time consuming, complex, and laborious. This disclosure aims to solve problems in the current state of the art, since currently up to 2.5 million pregnant women are put at risk of harmful treatments annually. This disclosure, for example, provides an add-on device to toilets to enable mass pregnancy screening in emergency departments, urgent care facilities, and other locations by substantially automatically determining a woman's pregnancy status through the urine without requiring clinical staff intervention, creating a faster, lower cost, and more reliable process.

The following disclosure provides for an add-on device installable to existing equipment to indicate a presence of a detectable medical condition. For example, the disclosure may relate to a device installable on toilets that automatically tests urine for CKD and other conditions in minutes. By substantially automating a process that requires little input from non-laboratory staff, devices provided by this disclosure reduce the required training and technology knowledge needed to operate a point-of-care testing (PoCT) device. One or more of the devices provided by this disclosure may enable reliable testing anywhere there is a toilet, such as in a physician's office, a retail walk-in clinic, a screening van, emergency department, urgent care facility, or other care areas, such as in a hospital.

Additionally, the components and operations of this disclosure may speed up the urine testing process per patient to less than 5 minutes. This time is a substantial improvement over the current industry standard, which is believed to be about 65 minutes from the time the patient arrives at the waiting room until clinical action is first taken.

The components and operations of this disclosure may increase detection of kidney stone development, for example, by analyzing pH. The testing may determine diet effectiveness, type of kidney stone developing, and other factors. The testing provided by this disclosure may eliminate unnecessary trips to a urologist for wasteful scans to rule out kidney stone. Similarly, automated urinalysis to detect urinary tract infections (UTI) may be performed at pharmacies, clinics, and offices of offsite nurse practitioners who can prescribe antibiotics (for UTI). This can be attractive for patients who do not want to pay a high deductible to go to a physician's office.

Additionally, the components and operations of this disclosure may be used to perform drug screening. In the age of heroin usage and increasing addiction, more kids and adults are overdosing on drugs. Similarly, emergency department, urgent care, and other drug screening may be facilitated, potentially allowing for screening of everyone as they come in.

Additionally, the components and operations of this disclosure may be used to screen for diabetic conditions. In the example of diabetic CKD, urine may be screened for urine microalbumin, such as creatinine for diabetes. This may change the management for angiotensin-converting-enzyme (ACE) inhibitors, which are traditionally sent to a lab for testing. In another example of borderline diabetics, urine may be screened for glucose to see if a patient is developing diabetes. The testing provided by this disclosure may provide at-home monitoring of glucose in urine, which may improve patient satisfaction because they no longer need a daily blood prick.

Diabetes patients are supposed to blood prick themselves about 4-6 times per day. This inconvenience may lead to patients with diabetes tending not to blood prick themselves because they feel they can “sense” when their blood sugar is low or high. When these patients “sense” that their blood sugar is low or high, they blood prick themselves to get a quantitative blood glucose measurement to determine how much medication they should take to increase or decrease their blood glucose levels. “Sensing” can lead to inaccuracies that can have adverse clinical outcomes. For these type of patients, a passive at-home monitoring device, like one provided by this disclosure, for urine glucose could potentially avoid these inaccuracies from “sensing.” From another perspective, children with diabetes and newly diagnosed diabetes are not very good at “sensing” their blood sugar levels, so having a passive at-home monitoring device for urine glucose could be beneficial to them.

Additionally, the components and operations of this disclosure may be used for detection of chronic diseases. Currently, patients need to titrate up and titrate down treatment. This disclosure provides a technique to test metabolites in urine to determine current titration level, which may reduce epilepsy, resulting seizures, and minimize hospital stay caused by these seizures.

Additionally, the components and operations of this disclosure may be used for monitoring medication adherence, in particular for cardiovascular purposes. Currently, physicians rely on patients to provide information about whether they are adhering to their medication. Now, physicians may more efficiently have patients test their urine for biomarkers that may be used to check medication adherence. This disclosure may further automate urine testing in either a clinical setting or the home setting to test whether the medication affects urine biomarkers.

Additionally, the components and operations of this disclosure may be used for at-home monitoring of patients for particular biomarkers. Currently, physicians may prescribe a treatment to a patient, but will not be able to track how effective the treatment is with a high frequency. Patients need to periodically go back to the clinical setting, so the physician and/or clinician can conduct a urine and/or blood test to look for increases in the concentration of a biomarker or combination of biomarkers, which is an indication that treatment is not effective and needs modification. Between patient visits to the clinical setting, time lags can occur on the order of days, weeks, and months between the patient undergoes treatment at home and when the physician measures treatment efficacy in the clinical setting.

With the components and the operations of this disclosure, physicians and/or clinicians may prescribe patients to install the system in their homes, in order to passively track over time with high frequency the concentration of one or many urinary biomarkers that are indications of disease progression and treatment efficacy. The system may track the concentration of urinary biomarkers whenever the patient urinates into the toilet at home. The system will securely send the test results to the clinical setting for the physician and/or clinicians to review the effectiveness of the treatment and/or the disease progression. The system will also analyze the trend over time of the concentration of the biomarker(s) compared to baseline biomarker concentration(s) unique to each patient. As an example, if the change in concentration of a specific biomarker exceeds a threshold compared to the baseline biomarker concentration, the system will automatically detect this trend and notify the patient and clinicians of treatment ineffectiveness and the disease progression.

Additionally, the components and operations of this disclosure may be used for performing general urinalysis tests. Currently, physicians rely on dipstick tests and microscopic tests to analyze the levels or evidence of glucose, bilirubin, ketone, specific gravity, blood, pH, protein, urobilinogen, nitrite, and leukocyte esterase in urine. This disclosure provides a technique to test or measure the general urinalysis assays.

In one aspect, the disclosure is directed to a system. The system includes a collection container configured to collect a urine sample from a patient. The system also includes a plurality of test strips configured to indicate a condition of the patient when exposed to the urine sample. Further, the system includes a fluid transportation system. The fluid transportation system is configured to transport a portion of the urine sample from the collection container to a first test strip of the plurality of test strips at a predetermined position relative to the collection container. The fluid transportation system is also configured to expose the first test strip to the portion of the urine sample. Further, the fluid transportation system is configured to deliver fresh water or another cleaning solution to the collection container to clean the collection container. In addition, the system includes a sensor configured to capture an image of the first test strip exposed to the portion of the urine sample when the first test strip is near the sensor. The image indicates the condition of the patient. Still further, the system includes a computing device configured to analyze the image of the first test strip captured by the sensor in order to determine the condition of the patient. Yet further, the system includes a motor. The motor is configured to position the first test strip near the sensor after the first test strip is exposed to the portion of the urine sample. The motor is also configured to position a second test strip of the plurality of test strips at the predetermined position after the first test strip is exposed to the portion of the urine sample.

In another aspect, the disclosure is directed to a method. The method includes collecting a urine sample from a patient in a collection container. The method also includes transporting a portion of the urine sample from the collection container to a predetermined position relative to the collection container using a fluid transportation system. Further, the method includes exposing, by the fluid transportation system, a first test strip to the portion of the urine sample. The first test strip is one of a plurality of test strips configured to indicate a condition of the patient when exposed to the sample. In addition, the method includes delivering, by the fluid transportation system, fresh water or another cleaning solution to the collection container to clean the collection container. Still further, the method includes positioning, by a motor, the first test strip near a sensor. Even further, the method includes capturing an image of the first test strip using the sensor. The image indicates the condition of the patient. Yet further, the method includes positioning, by the motor, a second test strip of the plurality of test strips at the predetermined position. Even still further, the method includes analyzing, by a computing device, the image of the first test strip in order to determine the condition of the patient.

In yet another aspect, the disclosure is directed to a replaceable cartridge. In some embodiments, the replaceable cartridge includes opposing reels. The replaceable cartridge also includes a plurality of test strips located on a belt that spans the opposing reels and configured to indicate a condition of a patient when exposed to a urine sample from the patient. The opposing reels are rotatable in order to move the belt and reposition the plurality of test strips. The test strips are spaced sufficiently far apart from one another on the belt such that a portion of the urine sample may be dispensed on one of the test strips without getting any of the urine sample on other test strips. In some embodiments, the replaceable cartridge may include stacks of test strips that are stacked vertically. The stack of test strips is housed in a cartridge with a slit at the bottom to pull out a test strip. In some embodiments, the replaceable cartridge may include a circular or elliptical carousel where test strips are rotated into the appropriate position.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, and aspects described by this disclosure will become more apparent from the following detailed description, along with the accompanying drawings in which like numerals represent like components.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1 is an illustration of the sample collection mechanism mounted on a toilet

FIG. 2 is an illustration of a patient scanning a wristband on a scanner on a separate mechanism of the diagnostic device.

FIG. 3 is an illustration of how the toilet seat component of the sample collection mechanism is raised or lowered based on the gender of the patient scanning the wristband on the device.

FIG. 4 is an illustration of a sterile collection cup that has been opened.

FIG. 5 is an illustration of where the sterile collection cup is placed relative to the sterile collection mechanism.

FIG. 6 is a top plan view of the sample collection mechanism as described in this disclosure.

FIG. 7 is a top plan view of the sample collection mechanism focusing on the vacuum holes at the bottom of the mechanism.

FIG. 8 is a top plan view of the film rolled on top of the sample collection mechanism.

FIG. 9 is a top plan view of the film unrolled to show the film with sodium polyacrylate strips and dissolvable plastic spaced at discrete intervals.

FIG. 10 is a side view of the entire sample collection mechanism, showing the seat, the film, the guide wire, and the rigid contoured base.

FIG. 11 is a side view of the sample collection mechanism as mounted on the toilet.

FIG. 12 is an illustration of the sample collection mechanism being raised to transfer the urine into the sterile collection cup and the diagnostic device.

FIG. 13 is a front view of the raised sample collection mechanism containing the sample before the sample dissolvable stoppers have dissolved.

FIG. 14 is a front view of the raised sample collection mechanism dispensing the sample after the sample dissolvable stoppers have dissolved.

FIG. 15 is an illustration of the full system after the sample collection mechanism has been lowered.

FIG. 16 is an illustration of the film being rolled over to be disposed as waste after the sample has been collected.

FIG. 17 is an illustration of a filled sterile collection cup being closed with its lid.

FIG. 18 is an illustration of the full system as the sample is being brought to a diagnostic device.

FIG. 19 is an illustration of the sample collection mechanism.

FIG. 20 is an illustration of the raised sample.

FIG. 21 is an illustration of the collection mechanism and diagnostic device installed on the toilet.

FIG. 22 is an illustration of the collection mechanism fully lowered onto the toilet.

FIG. 23 is a side-view illustration of the predefined location for the urine collector cup.

FIG. 24 is an overhead illustration of the predefined location for the urine collector cup.

FIG. 25 is a close-up illustration of the dispensing of urine from the urine collector to the urine collection cup.

FIG. 26 is an illustration of the dispensing of urine from the urine collector to the urine collection cup.

FIG. 27 is an illustration of the device installed in a bathroom.

FIG. 27A is a process flow for using the device

FIG. 28 is an illustration of one example of the device mounted on the toilet with an external film cartridge.

FIG. 29A is an illustration of one example of the device mounted on the toilet.

FIG. 29B is an illustration of one example of the device mounted on the toilet.

FIG. 30 is a process flow for using the device.

FIG. 31 is an illustration of one example of the device as a stand-alone device to the toilet.

FIG. 32 is an illustration of one example of the device without the collection mechanism.

FIG. 33 is an illustration of one example of the device with mobile/portable capabilities.

FIG. 34 is an illustration of one example of the device as a stand-alone device.

FIG. 34A is a process flow for using the stand-alone device.

FIG. 35 is an illustration of one example of the device without the urine collection mechanism.

FIG. 35A is a process flow for using the device without the urine collection mechanism.

FIG. 36 is an illustration of a urine collection sub-system.

FIG. 37 is an illustration of a urine collection sub-system.

FIG. 38 is an illustration of a test strip cassette and automation.

FIG. 39A is an illustration of a pregnancy analysis sub-system.

FIG. 39B is an illustration of an image processing algorithm.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods may be arranged and combined in a wide variety of different configurations, all of which are contemplated herein.

Furthermore, the particular arrangements shown in the figures should not be viewed as limiting. It should be understood that other embodiments might include more or less of each element shown in a given figure. In addition, some of the illustrated elements may be combined or omitted. Similarly, an example embodiment may include elements that are not illustrated in the figures.

The following disclosure is provided to describe various embodiments of a medical diagnostic system. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

Various aspects of the present disclosure will now be described in detail, without limitation. In the following disclosure, a platform for a sample collection mechanism that may be used as part of a medical diagnostic system will be discussed. Those of skill in the art will appreciate alternative labeling of the sample collection mechanism as a collection platform, a urine collection platform, a sample collection platform, or other similar names. Skilled readers should not view the inclusion of any alternative labels as limiting in any way.

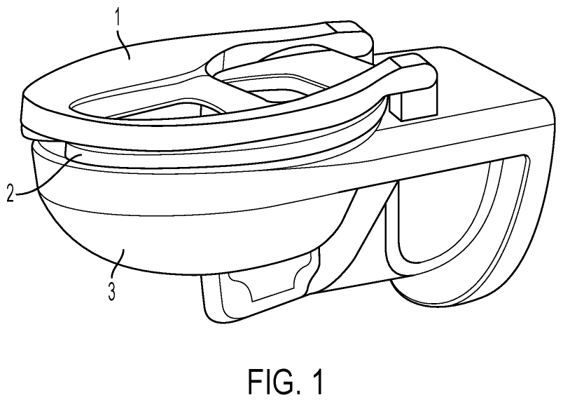

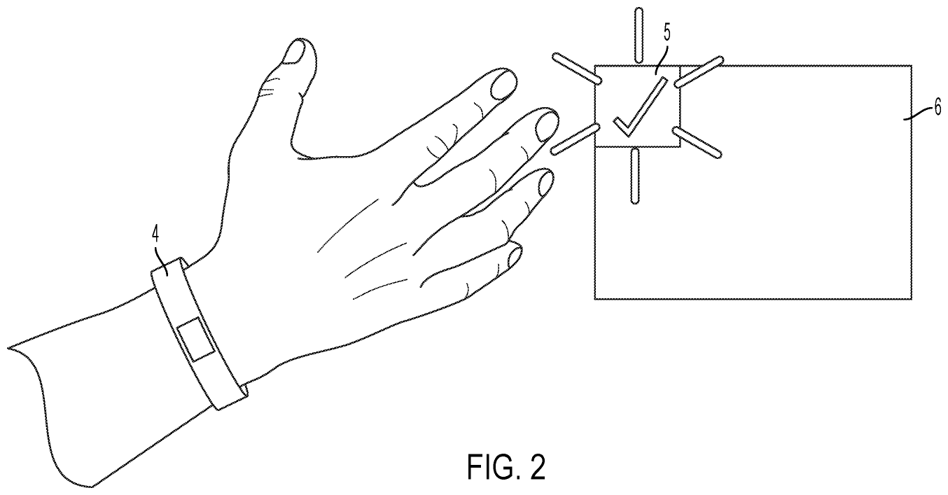

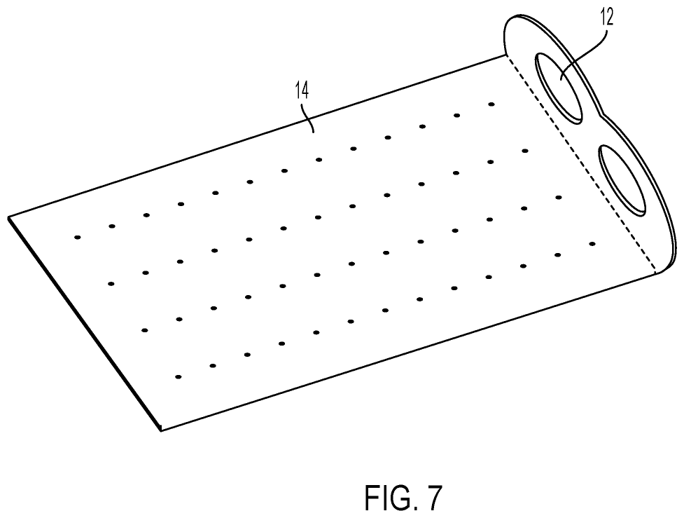

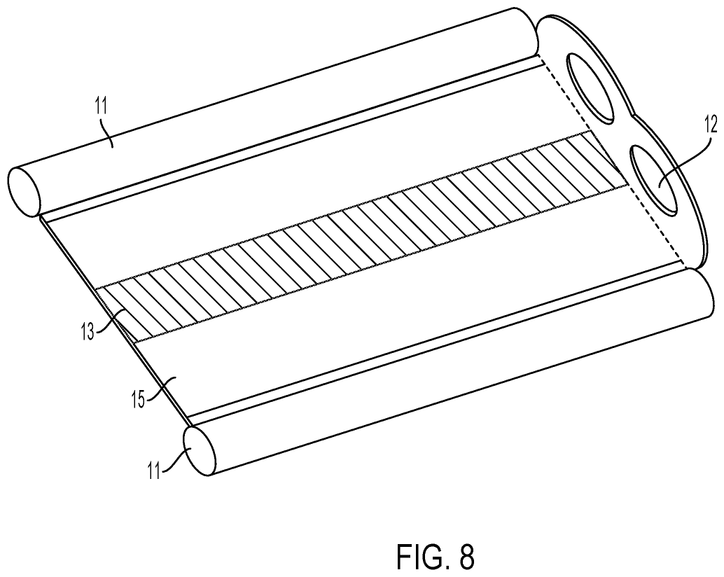

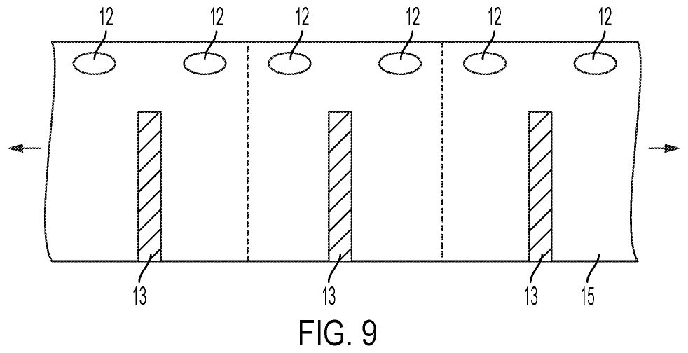

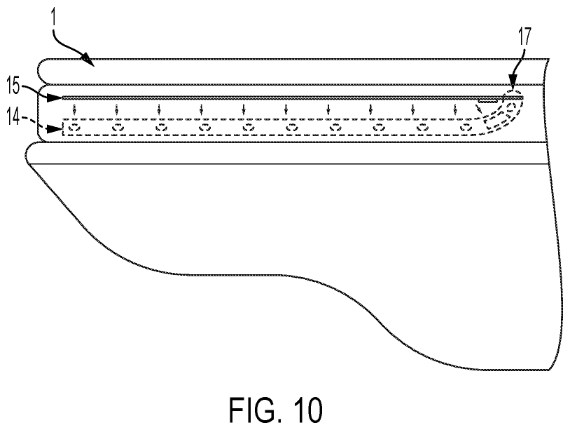

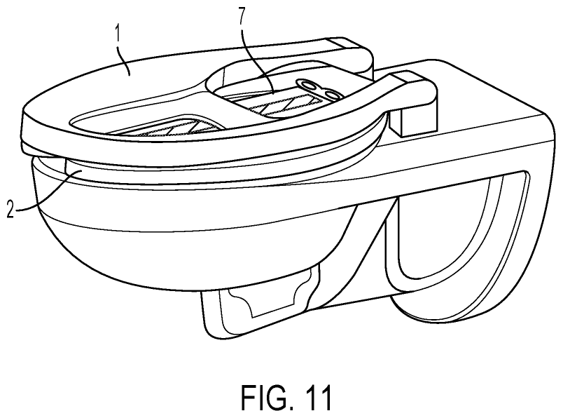

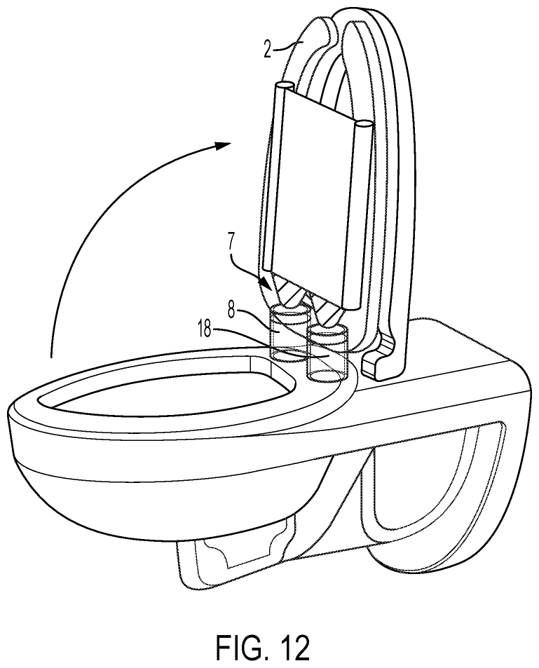

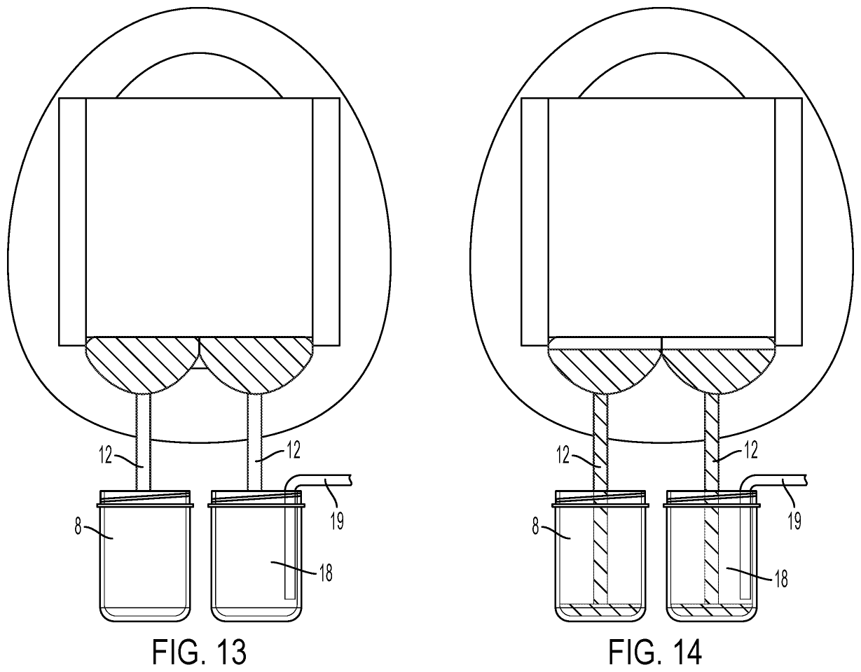

Referring now to FIGS. 1-18, the sample collection mechanism will now be discussed in more detail. The sample collection mechanism may include a urine collector, a roll of film, a rigid contoured base, a seat, a guide wire, and additional components that will be described in greater detail below. The sample collection mechanism may operate one or more of these components interactively to capture and transfer a sterile sample of urine.

Referring now to FIG. 1, the platform 2 will now be discussed in more detail. The platform 2 will be hinged together with a toilet seat 1 onto a toilet 3. The toilet 3 could be a residential or commercial toilet. The toilet 3 could be elongated or round. The toilet 3 could have different sizes of seat 1. The seat 1 will be hinged on top of the platform 2.

Referring now to FIG. 2, the patient scanning their wristband 4 will now be discussed in more detail. When a patient arrives at a medical office, as part of this process, he or she will receive a wristband 4 with a visual code. This visual code could be a barcode or a Quick Response (QR) code. When the patient is asked to provide a sample they will also receive a sterile collection cup 8 that is used to collect sterile urine for downstream lab testing and be led to the bathroom with the diagnostic device 6. In some embodiments, for example, the sterile collection cup 8 may be factory-sealed prior to use (e.g., the sterile collection cup 8 may have a tamper-proof seal). The sterile collection cup may be an industry-standard, factory-sealed collection cup or a cup customized for the devices described herein. To appropriately determine what tests need to be performed, the patient will scan the wristband 4 on the device 6, initiating the process. The device 6 will have a reader, such as a barcode reader or QR reader, which will detect the visual code on the wristband 4. The detected visual code will be interpreted by the device 6 using some reading algorithm to determine the appropriate tests. The device 6 may be located on the back of the toilet or on the side of the toilet higher than floor level. The scanner 5 on the device 6 will be accessible for all patients including patients in wheelchairs or patients who have difficulty lifting their arms or moving their wrists.

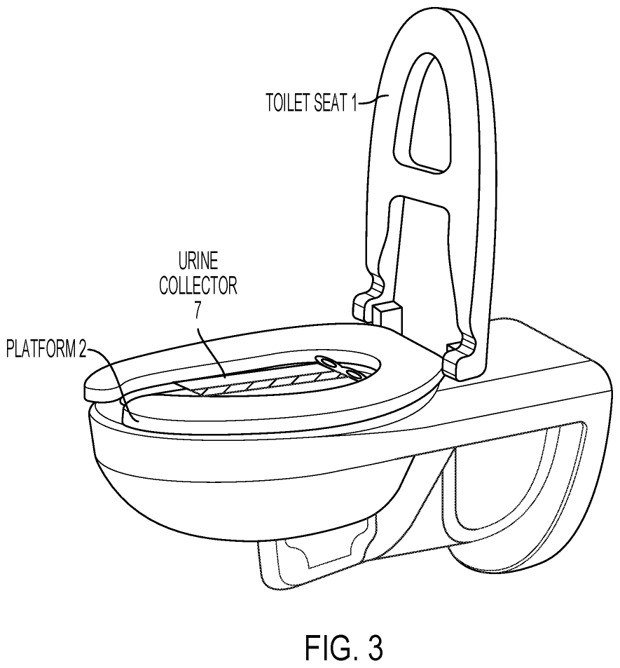

Referring now to FIG. 3, the raising and lowering of the seat 1 based on a patient's gender will now be discussed in more detail. After scanning the wristband 4, the seat 1 will either raise or lower based on the patient. If the patient is female, the seat 1 will be placed in a lowered position. If the patient is male, the seat 1 may be placed in a raised position. It is understood that, alternatively, the seat 1 may be placed in a lowered position for male patients. The seat 1 may be moved mechanically through some method such as an electric motor or it may be raised and lowered manually. The electric motor may be attached to the hinge point of the seat, for example. In addition, the urine collector (e.g., including the seat 1) may be actuated around the hinge point. The electric motor could be battery-powered or connected to an external power source (e.g., via a wall outlet). For example, a battery may be placed in the urine collection mechanism as part of a disposable component (e.g., the rolling film). The electric motor may be powered by the same power source used to power the urine analysis subsystem. Alternatively, the motor may be mechanically powered (e.g., rather than an electric motor). Such a mechanical motor may be powered by a manual crank, for example.

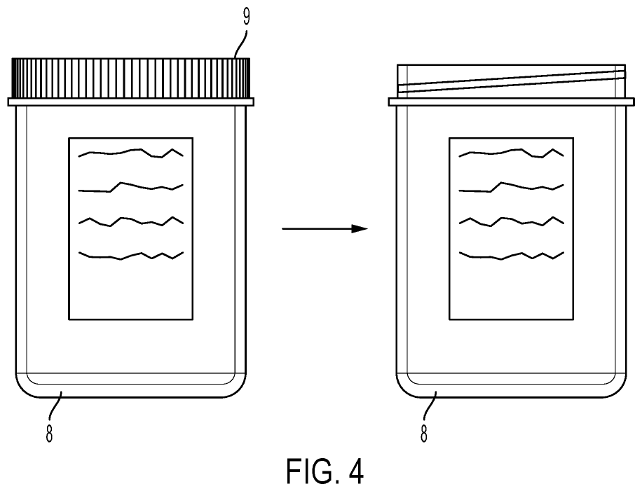

Referring now to FIG. 4, the sterile collection cup 8 will now be discussed in more detail. Prior to urinating into the urine collector 7, the patient will unscrew the cap 9 from the sterile collection cup 8. The sterile collection cup 8 collects urine for downstream lab testing. Downstream lab tests may include urine cultures or microscopic analysis. Additionally or alternatively, follow-up tests for urinanalysis or pregnancy may be performed to confirm test results produced by the device described herein. The cups may come with barcode stickers on them. The stickers may be peeled off to place on specimen tubes later on in the workflow for downstream tests. The patient may also scan the visual code on the collection cup 8 onto the device 6. The device 6 may identify the visual code and match the visual code to the same code on the patient's bracelet 4. The barcode data may then be sent to the electronic health record along with the point-of-care test results to be appended to the patient ID. In various embodiments, one-dimensional, two-dimensional, and/or three-dimensional barcodes may be used. The device 6 may connect to an electronic health record via a WIFI connection, a BLUETOOTH connection, and/or an ETHERNET connection. Further, in some embodiments, the sterile collection cup 8 may be replaced or augmented by a test tube or test vial (e.g., having a volume of 5.0 mL or less), such as a custom test tube or test vial designed specifically based on the device 6.

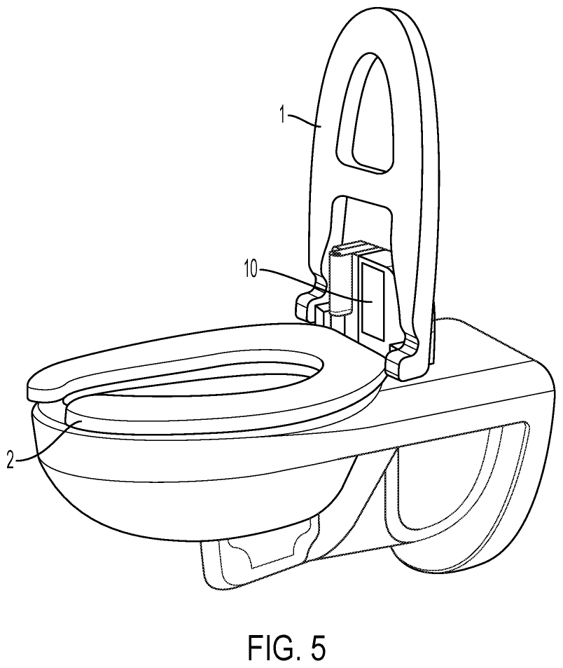

Referring now to FIG. 5, the placement of the sterile collection cup 8 will be now be discussed in more detail. After unscrewing the cap 9 from the sterile collection cup 8, the sterile collection cup 8 will be placed in the back of the platform 2 in a predefined location 10. The predefined location 10 could be identified by a visual aid, such as words stating “PLACE CUP HERE,” an image showing a picture of the sterile collection cup, or by a visual aid using one or more previously defined methods.

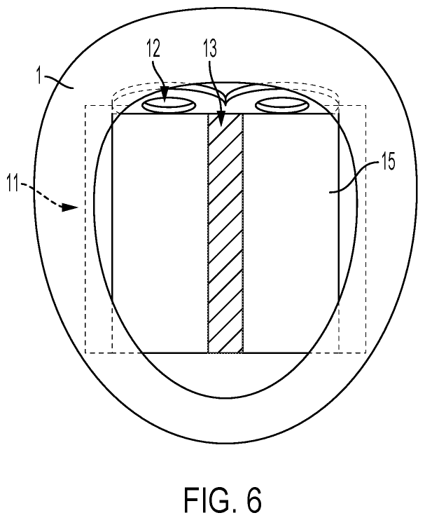

Referring now to FIGS. 6-11, the urine collector 7 of the platform 2 will be discussed in more detail. The platform 2 contains the rollers 11, a rigid contoured base 14, and the film 15 (collectively referred to as “the collector” 7). The collector 7 could also be inserted on the side of the platform 2, using a handle or a push-through mechanism. The collector 7 could also be inserted from the top of the platform 2, using slots in the platform 2 that may fit the collector 7. The collector 7 could rotate to switch from one side to another. The rotation may occur quickly or at a slow pace. The collector 7 could flip on its axis. The collector 7 could have a retractable lid or a retractable bottom. The collector 7 could flip into the toilet from outside the toilet. The collector 7 could be connected to the sterile collection cup 8. The collector 7 could have clasps on the perimeter to latch the collector 7 to the platform 2. The collector 7 could have a syringe or opening at the bottom. The collector 7 could have an anti-microbial coating (e.g., located on the rigid central platform) to keep the collector 7 sterile and/or minimize bacteria build-up. The collector 7 may additionally or alternatively have a hydrophobic coating. The film 15 may be shaped to allow for clean catch by having a trough 31 in the center to capture the initial stream of urine. The film 15 may be replaced once a roll of film has been completely used. Alternatively, the film 15 may have an absorbent strip 13 such as a strip of sodium polyacrylate, at specific intervals on the film 15 to capture and hold the initial stream of urine. In alternate embodiments, other materials besides sodium polyacrylate may be used for the absorbent strip 13 (e.g., superabsorbent polymers, water-absorbing polymers, hydrogels, or a liquid-absorbing polymer that swells but does not disintegrate when exposed to water). For example, the absorbent strip 13 may include potassium acrylate, polyacrylamide copolymer, ethylene maleic anhydride copolymer, cross-linked carboxymethylcellulose, polyvinyl alcohol copolymers, cross-linked polyethylene oxide, starch grafted copolymer of polyacrylonitrile, superabsorbent polymers derived from polysaccharides and proteins, and/or soy protein/poly(acrylic acid). The absorbent strips 13 may be spaced at discrete intervals. In some embodiments, the absorbent strips 13 may be in discrete strips on the film. Alternatively, the absorbent strips 13 may be scattered and spread throughout film. The film 15 may be rolled inside a disposable reel-to-reel cartridge. One side of the cartridge may have fresh film, while the other side receives used film. Clinical staff may throw out the cartridge once per day. Enough film may be contained in a cartridge to service 100 patients, in some embodiments. A film sub-system unit may repeats throughout overall film. The film 15 (and associated film sub-system) may include holes/dissolvable stoppers 12 or it may have shapes made of water-soluble plastic such as polyvinyl alcohol polymers (e.g., PVOH, PVA, or PVAI) placed in the rear of the collector 7. Alternatively, the film 15 may have ramps 20 that are usable to pour the urine into the desired receptacles. The plastic shaped into holes that include dissolvable stoppers 12 that will dissolve when in contact with urine. The dissolvable stoppers 12 are spaced at discrete intervals. The urine collector 7 consists of a film 15 that is rolled across the rigid contoured base 14. The back of the film 15 may be stabilized, for example by using guide wire 17. In the embodiment with a guide wire 17, the film 15 may be hooked onto a guide wire 17 to keep the film in place. The guide wire 17 may be made of metal or plastic and/or may be placed towards the hinge of the toilet seat. There may be hooks on the film 15. Additionally or alternatively, the film 15 may have a loop that connects to the guide wire 17 wire upon installation. When a new sheet of film 15 is rolled over the collector 7, the guide wire 17 makes sure that the new film 15 stays connected to the back of the toilet. The rigid contoured base 14 may hold down the film 15, using forces like vacuum holes or electrostatic force. In the embodiment with vacuum holes, the vacuum may be turned off to roll the film over the rigid contoured base 14 and turned back on once the film 15 has rolled on. The vacuum holes may be drilled into the central platform during manufacturing. The vacuum pump may create a vacuum through the vacuum holes that hold the film down against the central platform. The vacuum pump may be powered by battery (e.g., by a battery inserted into the urine collection mechanism) or plugged into a wall socket (e.g., to use the same power source as the rest of the device). Alternatively, the rigid contoured base 14 may have ridges to trap some urine. The ridges may be contoured to be concave so that when the seat is upright, the seat holds the urine. The ridges may be located on an end closer to the toilet tank or the flush valve, for example. The back side of the collector 7 is curved up in order to hold the urine when the collector 7 is raised. The curvature of the collector 7 may be less than the curvature of a standard toilet bowl. A standardized curvature may be selected for use across all toilet bowls or a curvature designed for a particular toilet bowl may be selected. Some embodiments may include dissolvable stoppers 12 made of two water-soluble plastic polyvinyl alcohol polymers (e.g., PVOH, PVA, or PVAI) as part of the film 15 that are positioned at the back side of the collector 7.

The process starts when the patient urinates into the collector 7. As the patient urinates into the collector 7, any urine that exceeds the volume of the collector 7 may overflow into the toilet bowl. The absorbent strips 13 capture the initial stream and become saturated. This enables a clean catch to be performed as the initial urine is in at least one of the absorbent strips 13. The film 15 rolls over the rigid contoured base 14 to maintain a smooth curved shape in the middle of the toilet. When the platform 2 raises, the urine collects via gravity above the two dissolvable stoppers 12 or ramps 20 in the back side of the collector 7. After some time such as 10 seconds, the urine dissolves the dissolvable stoppers 12 and drops via gravity into the sterile collection cup 8 for downstream lab tests and a fixed collection reservoir 18 connected to the diagnostic device 6. Patient pushes the flush handle on the toilet or activates the flush mechanism on the toilet. Delayed flush that is controlled by the device 6 occurs after the testing is complete.

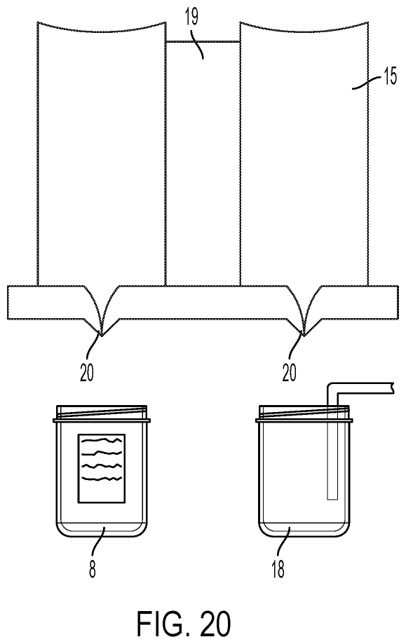

Referring now to FIGS. 12-14 and 20, the raising of the platform 2 post-flush will now be discussed in more detail. After the patient pushes the flush lever, the platform 2 will automatically rise. The urine will move towards the back of the urine collector 7 where the urine will be collected above plastic pockets, which may include dissolvable stoppers 12. The platform may be raised and/or lowered automatically and mechanically through an electric motor, in some embodiments. In some embodiments, such an electric motor may be powered by plugging into a wall outlet and/or by utilizing a battery (e.g., a battery configured to power the entire device). Alternatively, the platform may be raised and/or lowered manually (e.g., by a patient). There will be a urine collection reservoir 18 which has tubes 19 connected to the diagnostic device 6. In the embodiment with dissolvable stoppers, the dissolvable stoppers 12 at the bottom may dissolve in approximately ten seconds. After the dissolvable stoppers 12 dissolve, the urine drains into both the open sterile collection cup 8 and the urine collection reservoir 18 for the diagnostic device 6. In the embodiment with ramps 20, the urine may flow through the ramps and into the open sterile collection cup 8 and the urine collection reservoir 18.

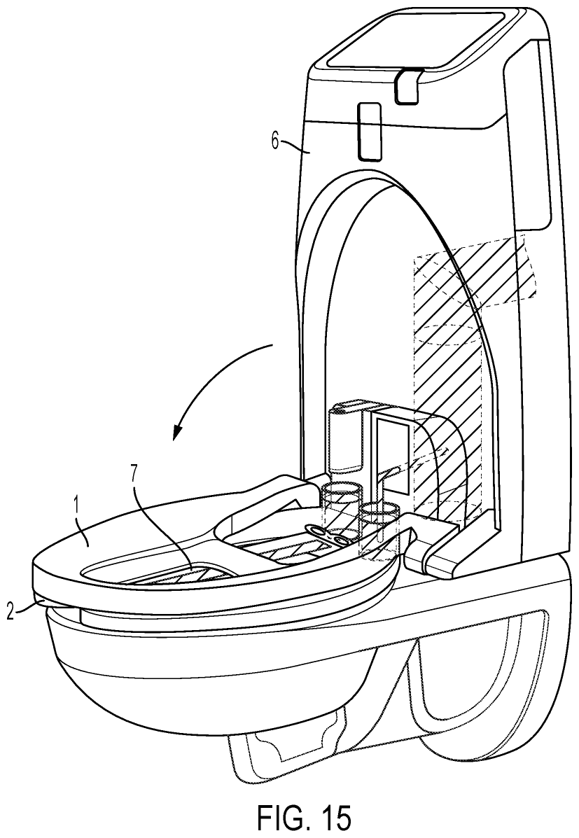

Referring now to FIG. 15, the lowering of the platform 2 will now be discussed in more detail. After the urine is fully drained, the platform 2 will be lowered to its original position. At this point, the toilet will flush out any waste.

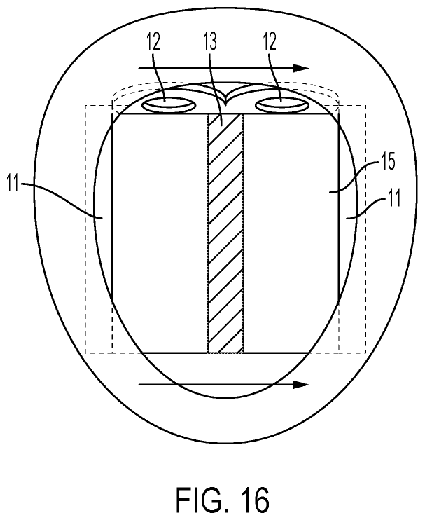

Referring now to FIG. 16, the rolling of the used film 15 will now be discussed in more detail. The used film 15 will be rolled towards the right, and a replacement film 15 will be rolled over the collector 7. The film 15 may be inside a disposable film cartridge. Clinical staff may dispose of the cartridge in a garbage receptacle once a day and replace with a new cartridge. Such a replacement may take place when the film 15 is used up during the day. For example, the device may notify clinical staff when the film 15 is used up, or predict when the film 15 is about to be used up and notify clinical staff that a replacement cartridge will soon be needed.

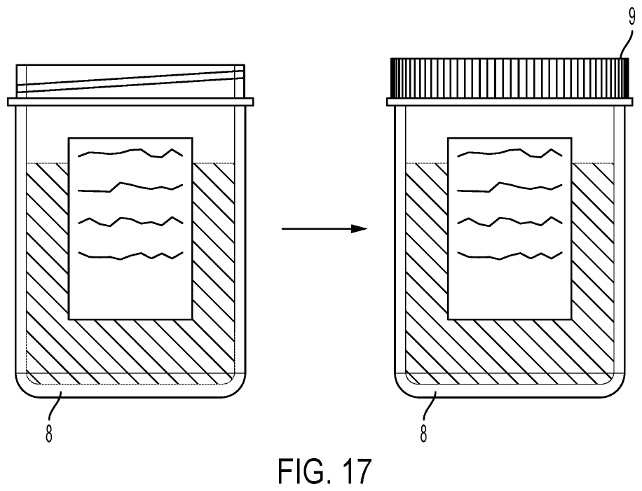

Referring now to FIG. 17, the filled sterile collection cup 8 will now be discussed in more detail. The sterile collection cup 8 will be partially filled. Patient will pick up the sterile collection cup 8, screw on the cap 9, leave the restroom, and place the sterile collection cup 8 in a designated area outside the restroom.

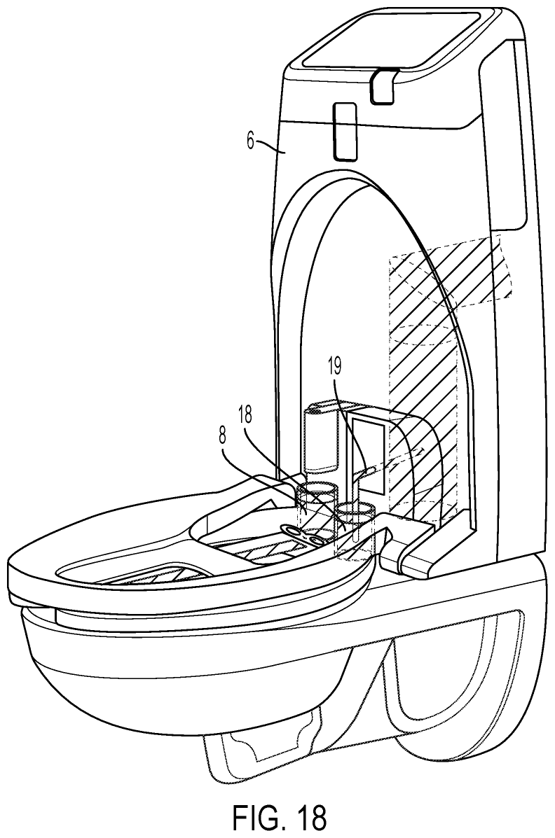

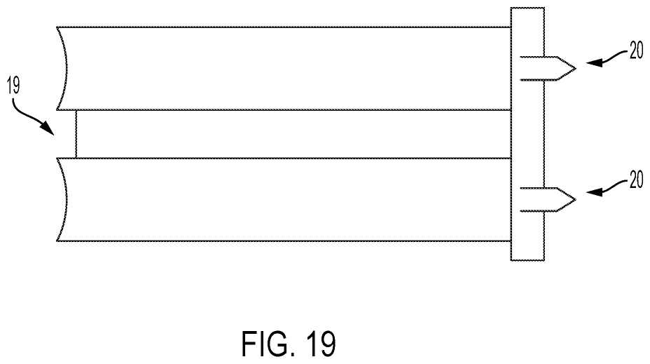

Referring now to FIG. 18, the interaction with a separate diagnostic device 6 will now be discussed. The urine is transferred from the urine collection reservoir 18 to the diagnostic device 6 via tubing 19 and the device 6 performs the urine analysis.

The analysis of the urine sample will now be discussed in more detail. The urine sample may be analyzed using hyperspectral imaging, multispectral imaging, or a spectrophotometer implementation in disclosures with or without the urine collection mechanism.

The analysis of the urine sample using a diagnostic device with a cassette will now be discussed in more detail. The cassette may hold a plurality of test strips. The cassette may have bleach powder to sterilize the entire diagnostic system, plus urine collection mechanism.

An alternative method of sterilizing the urine collector 7 will now be discussed in more detail. Another method to sterilize the urine collector 7 would be to use a light source like an ultra-violet light source.

A method of collecting the urine will now be discussed in more detail. A sponge or sponge-like material could be used to collect the urine. The sponge could absorb the urine and then release the urine by self-squeezing the sponge or by squeezing the sponge using a set of rollers that are covered with film.

Another method of collecting the urine will now be discussed in more detail. A non-contaminative material may be used to collect the urine such as water or a powder. The powder may melt as the urine hits the material.

Another method of collecting the urine will now be discussed in more detail. An inclined urine collector may be used. The urine may hit the inclined collector. Water may be flowing down the collector during urination. The urine may then be collected in a container. The container may be at the bottom of the incline or in the middle of the incline.

Another method of analyzing and collecting the urine will now be discussed in more detail. A sensor may be placed on the inside of the bowl of the toilet. The sensor may be installed inside the bowl of the toilet or the sensor may be attached to an arm on the side of the bowl. The urine may flow through the sensor and a result may be determined using a method, such as spectrophotometry. The sensor may have anti-microbial coating on the inside of the sensor and the outside of the sensor.

Another method of analyzing and collecting the urine will now be discussed in detail. Some embodiments may include a robotic arm with an open collection cup that uses visual sensors to find the stream of urine, capture the urine, and finally cap the collection cup.

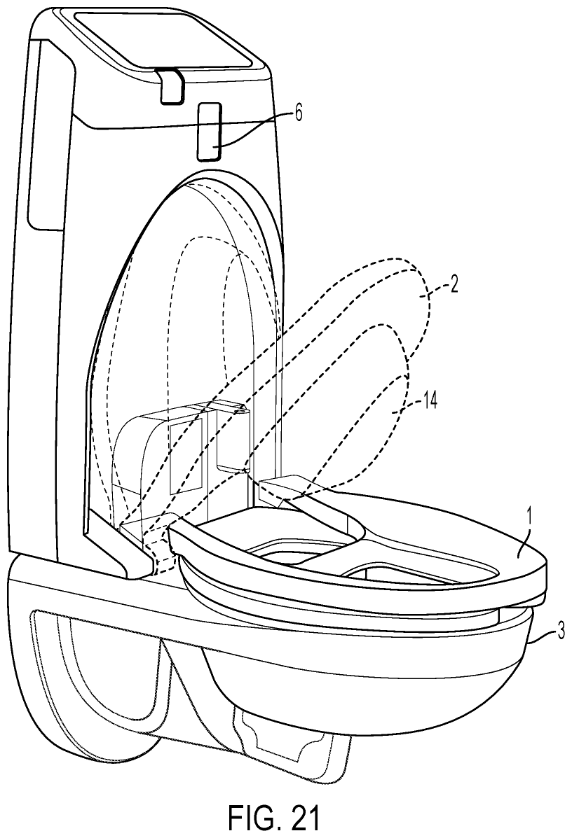

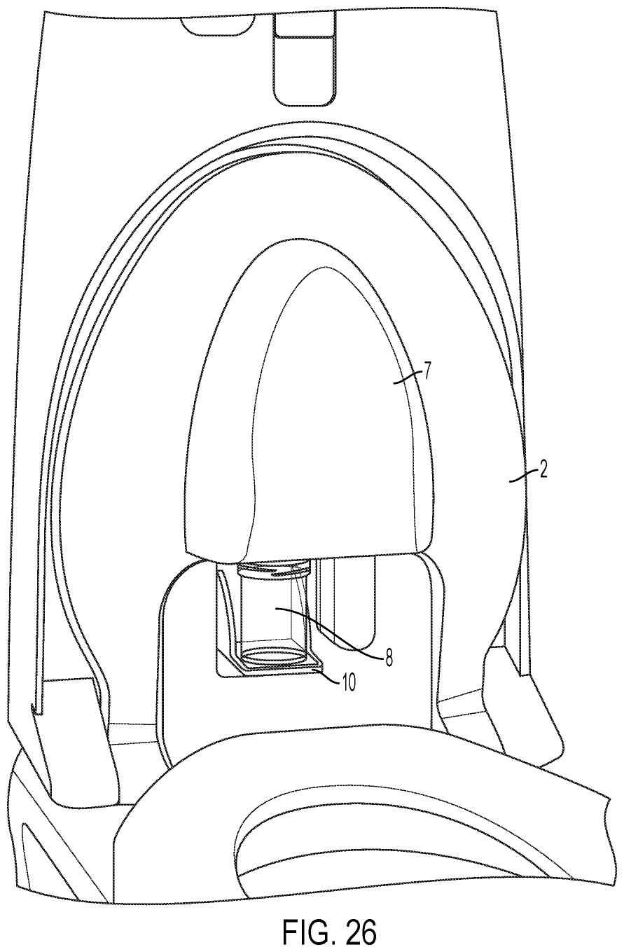

Referring now to FIG. 21-27, an embodiment demonstrating the platform 2 installed on the toilet 3 will now be discussed. In this embodiment, all components of the device are installed on the toilet 3.

Referring now to FIG. 21, the lowering of the urine collector 7 on the platform 2 onto the toilet 3 will now be discussed. The urine collector 7 may be automatically or manually lowered into the platform to allow a user to sit and urinate into the collector 7. The urine collector 7 may be lowered so that it is be placed on top of the seat 1. The embodiment also includes a scanner 5 and an instruction screen which demonstrates how to use the device. The demonstration may be visual or audible. In the visual embodiment, the visual demonstration may be static drawings or videos that show how to use the device. In the audio embodiment, the audio may be optionally enabled for users who cannot see or understand the visual demonstration. The scanner 5 may be any visual scanner including a barcode scanner, a camera, or a radio-frequency identification (RFID) scanner. The scanner 5 may be powered using electricity from the urine analysis subsystem, a power outlet, or a separate battery source. The scanner 5 may relay information to the urine analysis subsystem, which then sends information to the EHR through WIFI. The instruction screen may be a liquid-crystal displace (LCD), a light-emitting diode (LED) display, or a cathode ray tube (CRT) monitor. The instruction screen may also be a depiction of the steps on a sticker or another static visualization. The instruction screen may be mounted next to the toilet or on a wall behind the toilet. External speakers for audible instructions may be mounted within the monitor for visually impaired users. The speakers may be enabled by the user scanning the bracelet or by medical staff activating the speakers using a mobile application or manually activating the speakers prior to use. The instruction screen may be integrated onto the top of the device, in some embodiments. Further, speakers may be activated by users interacting with the instruction screen, in some embodiments.

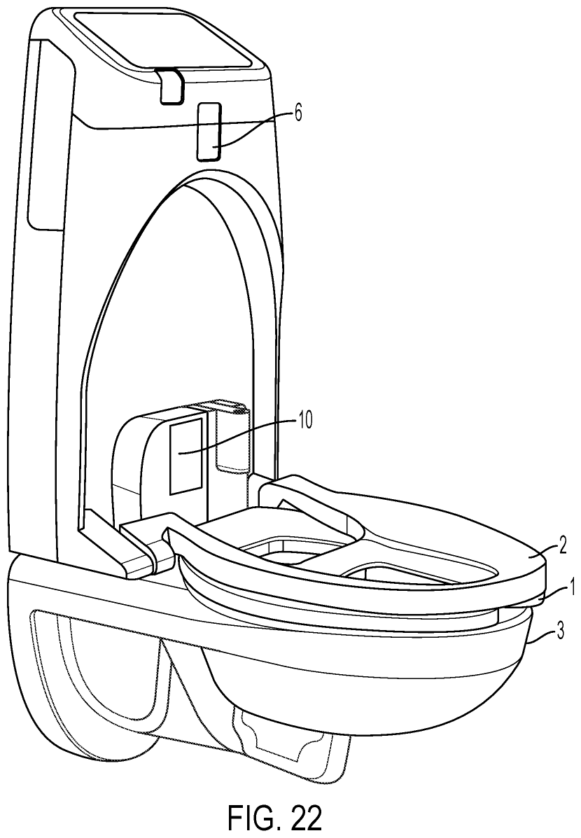

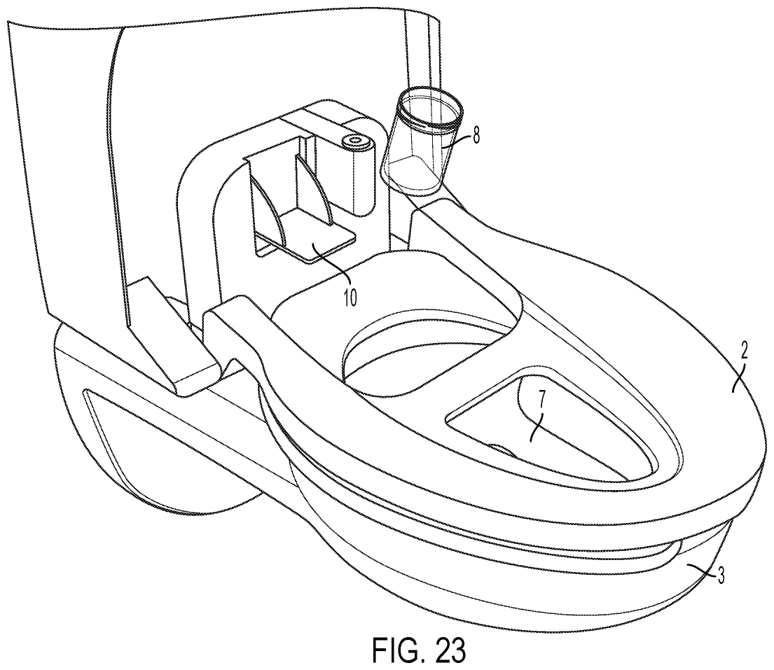

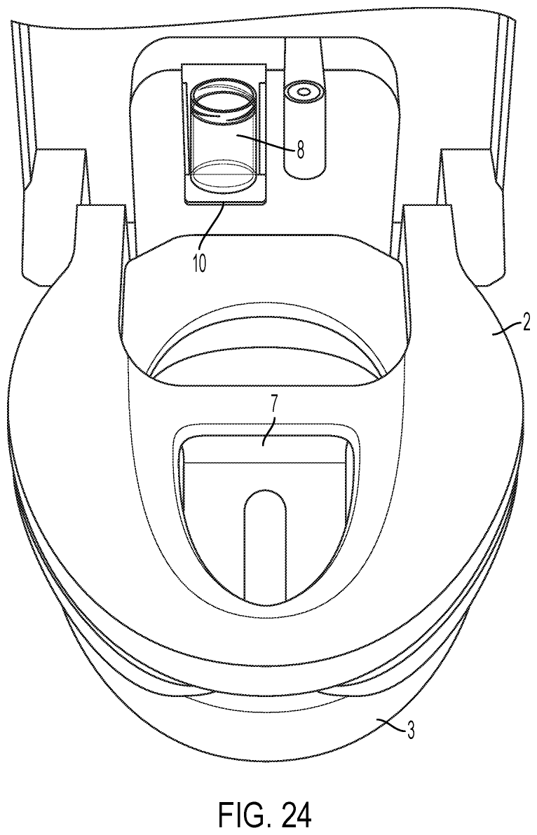

Referring now to FIG. 22, the embodiment where the platform 2 with the urine collector 7 is fully lowered into the toilet 3 will now be discussed. When the platform 2 is fully lowered onto the toilet 3, a predefined location 10 for the urine collection cup 8 is seen in a position where it is stowed away. The platform 2 may have an open space for disposal of toilet paper which is located toward the rear of the toilet. The predefined location 10 may be the size of an industry-standard urine collection cup without the lid on (e.g., 5.5 inches x 3.75 inches x 3.25 inches)

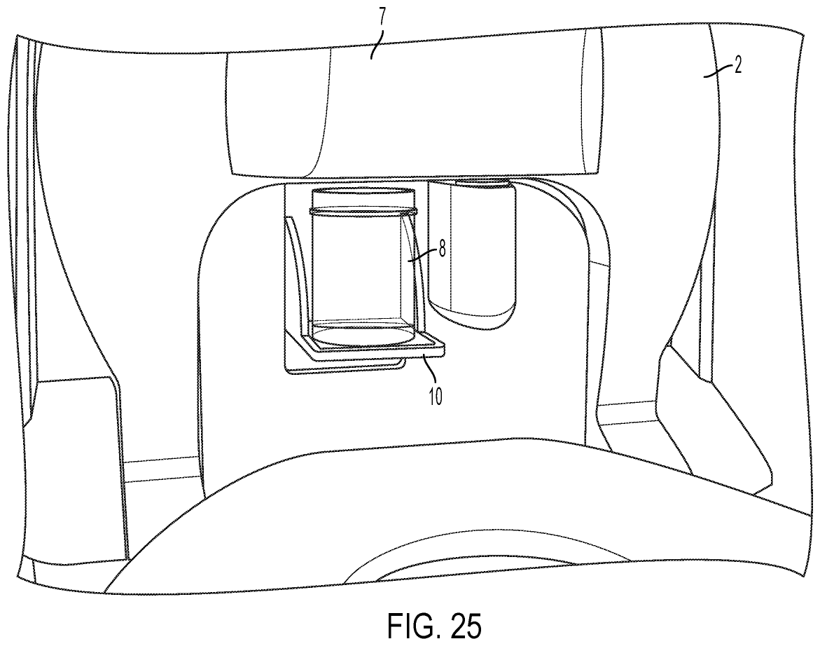

Referring now to FIG. 23-25, the predefined location 10 for the urine collection cup 8 will now be discussed. The predefined location 10 may be opened or closed automatically by the device so that the user may place the urine collection cup 8 into the predefined location 10. The predefined location 10 may be revealed using an electronic signal or a mechanical method. The predefined location 10 may be opened based on scanning the bracelet, manually by medical staff, or by mobile app by medical staff. The predefined location 10 may be permanently in the open position. The predefined location 10 could open and close through a door that opens downward, upward, or from the side. Such a door may be motor-powered and the motor may be connected to the diagnostic device 6. The instruction screen may instruct the patient to scan their wristband to activate the device when patient enters the restroom. Upon scanning, the predefined location 10 may open automatically

Referring now to FIGS. 25 and 26, the raising of the platform 2 to dispense the urine into the collection cup 8 will be discussed. The platform 2 is raised automatically to dispense the urine from the urine collector 7 into the collection cup 8. The platform 2 may be raised after the patient re-scans the barcode. Rescanning the barcode may also cause the toilet to flush. The instruction screen may instruct the patient to scan wristband when they are done urinating into the collector. The collector may then raise automatically after the patient has scanned the wristband.

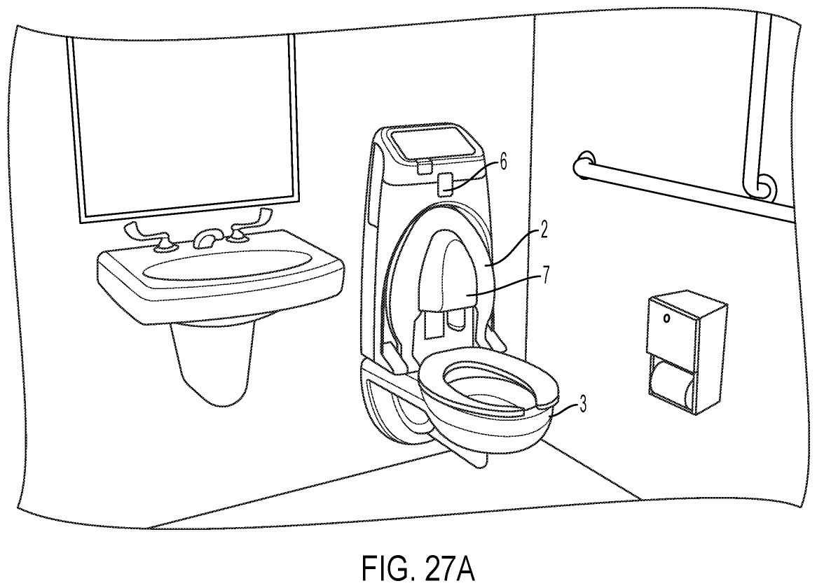

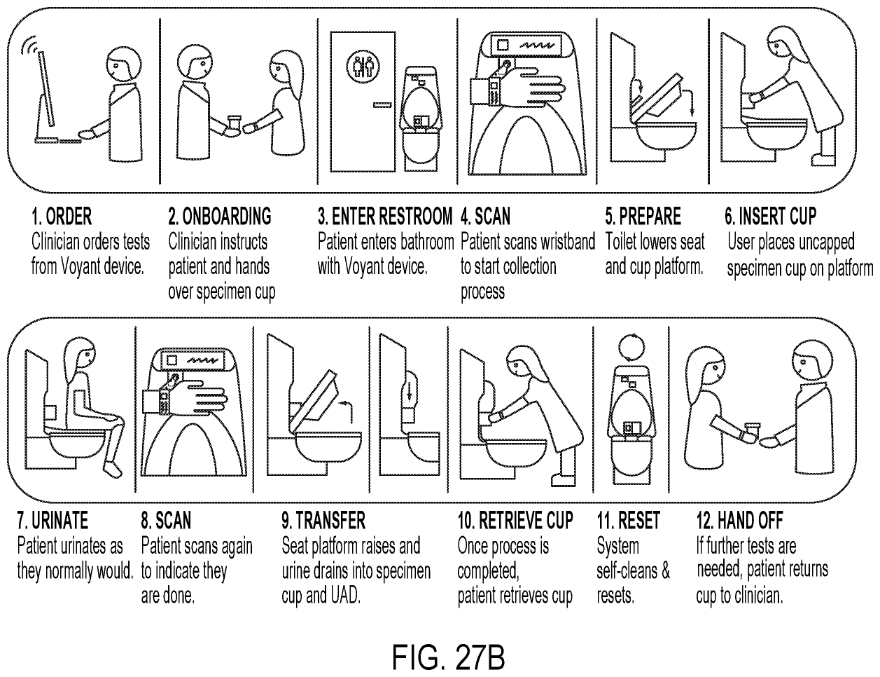

Referring now to FIG. 27, the device installed in the bathroom will be discussed. The device may be installed in the bathroom, even bathrooms that are designed for disabled patients.

Referring now to FIG. 27 A, the process flow for using the device mounted on the toilet will be discussed. The clinician orders tests from the device. The clinician then instructs the patient and hands over the specimen cup 8. The patient enters the bathroom with the device. The patient scans their barcode to start the process. The device lowers the platform. The patient inserts the cup 8 as instructed. The patient urinates as normal. The patient scans the barcode again to indicate they are done. The urine is transferred to the device 6 and the cup 8. The patient retrieves the cup 8. The system resets itself as it analyzes the urine. The patient hands off the sample to the clinician.

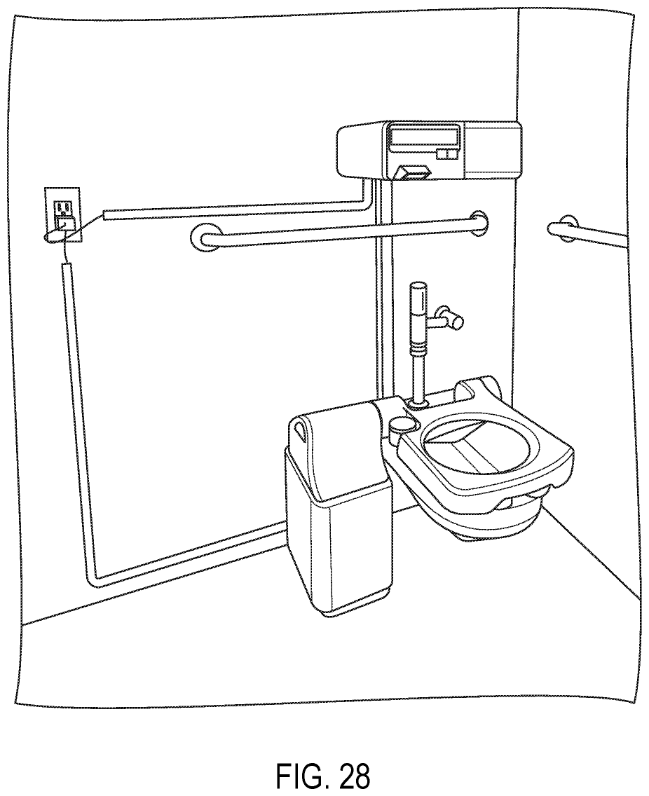

Referring now to FIG. 28, another example of the device mounted on the toilet will be discussed. Two component system consisting of a wall-mounted diagnostic device 6 and a urine collector 7 that attaches to a public toilet. The platform 2 and toilet seat are hinged together. The platform 2 hinges above the toilet seat to allow for toilet access when a sample is not needed. The user places the collection cup 8 into the platform 2. A disposable film 15 collects the urine sample. In alternate embodiments, more than one disposable film 15 may be used. Once the sample is collected and the platform 2 is raised, the urine sample transfers into the urine collector reservoir and the collection cup 8. When the platform 2 is lowered, the urine collector 7 resets and self-sanitizes as the next film 15 section rolls into place within platform 2. Users may remove the sample after the process is complete for further urine analysis testing. The diagnostic device 6 may be wall mounted, have a corded 120V AC power supply, include the patient ID scanner, a streamlined interface, a cartridge-based test strip, an optical test strip analyzer, on-board diagnostics, pump-driven fluid management, integrated fluid path sterilization, analysis/collection system control. A toilet flush interface, a platform interface (both fluid and electronic), and wireless electronic medical records (EMR) or electronic health records (EHR) connectivity. The collector 7 may be attached to a typical ED toilet, have corded 120 volt AC power supply, an analysis interface, contoured toilet seat interface, a position control mechanism with manual overdrive, a urine collection film 15 (UCF) cartridge with integrated sodium polyacrylate pads and soluble polymer ports, a UCF transport mechanism, a UCF vacuum system, a urine specimen cup interface, and a toilet paper bypass.

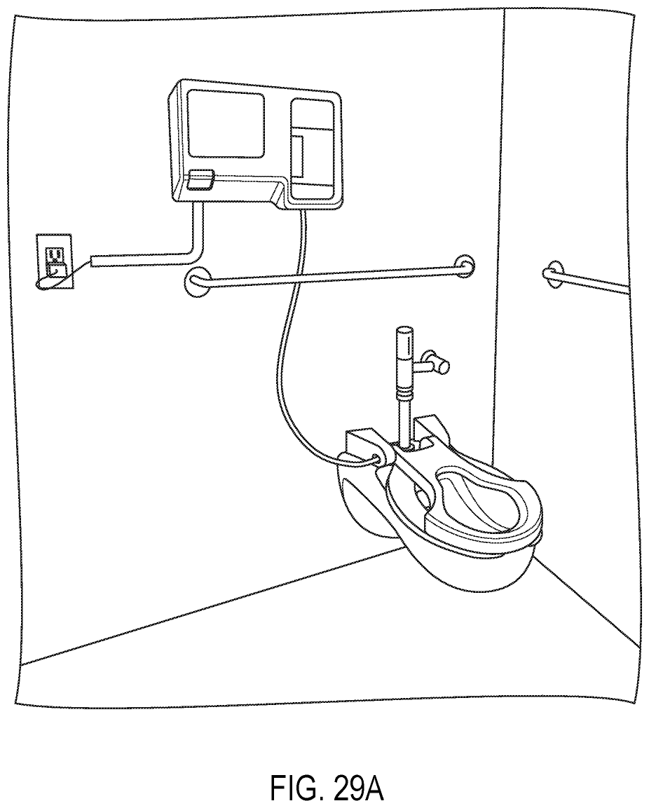

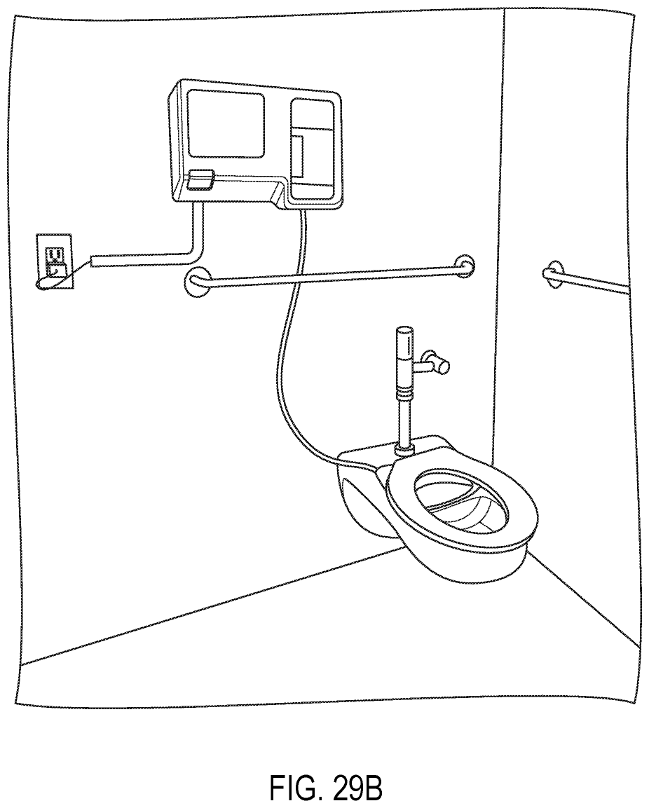

Referring now to FIG. 29A and FIG. 29B, another example of the device mounted on a toilet will be discussed. Two component system consisting of a wall-mounted diagnostic device 6 and manually positioned urine collector 7 mated to a typical toilet. The diagnostic device 6 provides primary user interface and system control. Touchscreen interface provides patient with task-based walk-through of process. The diagnostic device 6 accommodates placement of typical urine specimen cup. The urine collector 7 provides capture of first void urine and reserves midstream flow for collection by the diagnostic device 6. Urine is transferred from the platform 2 to the diagnostic device 6 for point-of-care analysis and specimen collection for downstream testing. Integrated sterilization refreshes system between uses. The design of the collector 7 may vary as demonstrated in the different designs in FIG. 29A and FIG. 29B.

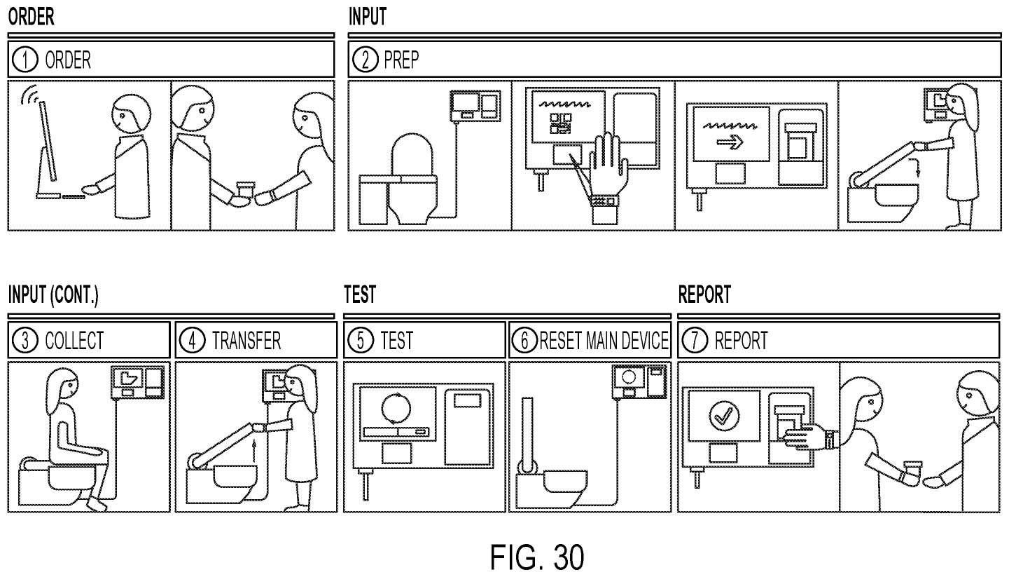

Referring now to FIG. 30, a process flow of how the patient will interact with the device is shown. The medical staff will order a test and provide the patient with a cup for the urine sample. The patient will follow the prompts on the instructions screen, prompting the patient to scan their barcode to the device, insert the cup and lift the seat. The patient will then urinate into the urine collector and transfer the urine into the cup by lifting the seat. The device will then test the results and reset the main device. The device will report the results to the EHR and provide the patient with the cup with sterile urine, which the patient will provide to the medical staff

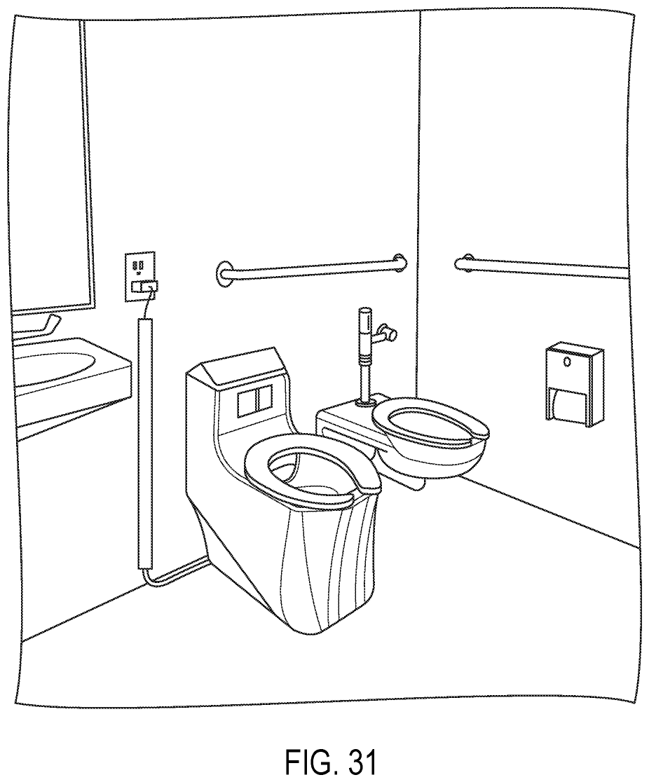

Referring now to FIG. 31, another example of the device will be shown. The self-contained system includes the urinalysis engine, integrated specimen collection, and wireless EMR connectivity. A refillable reservoir provides clean water for flush and system sterilization functions. A second reservoir collects excess waste for disposal. The device may be a stationary all-in-one system. The device may connect to the EMR through WIFI or BLUETOOTH. A WIFI-enabled microprocessor (e.g., a RASPBERRY PI) in the device may connect to the EMR using WIFI, for example. The device may also be connected to the flushometer of the toilet. The device may automatically pump some water from the flushometer into the clean water reservoir. The device may also automatically pump the clean water from this reservoir into the rest of the device to clean the device. The waste reservoir may be connected via tubing into the toilet bowl. The waste urine and fluids may be pumped automatically by the device from this reservoir into the toilet bowl.

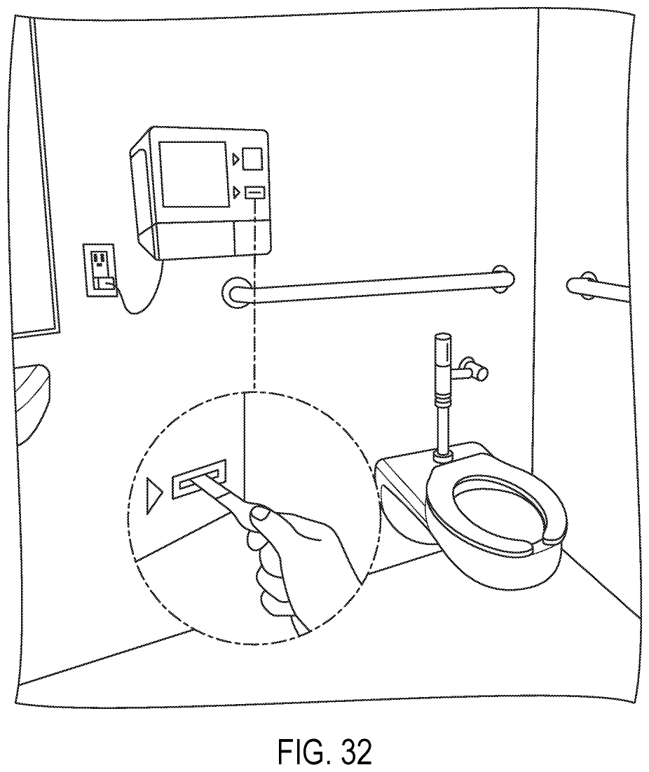

Referring now to FIG. 32, another example of the device will be shown. Self-contained diagnostic device 6 containing a urinalysis engine as described herein, integrated sterilization and wireless EMR connectivity. Patient uses proprietary test strip collecting midstream urine sample. Patient inserts sample into device for analysis and continues on. The diagnostic device 6 collects the sample, performs analysis and transmits results. Device is emptied and refreshed at regular intervals.

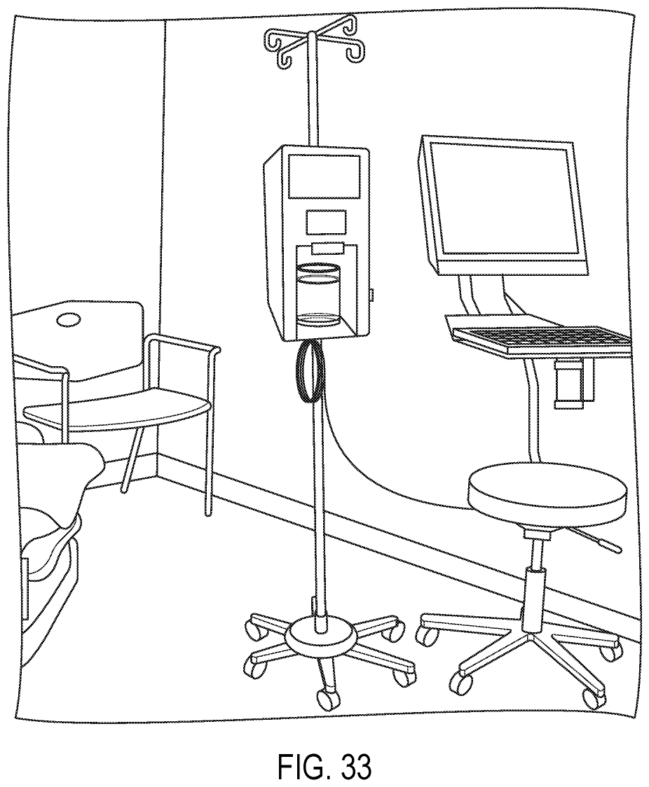

Referring now to FIG. 33, another example of the device is shown. Self-contained diagnostic device 6 containing a urinalysis engine as described herein, integrated sterilization and wireless EMR connectivity. Patient uses urine specimen cup proprietary cap to collect midstream urine sample. Patient inserts sample into device for analysis. The diagnostic device 6 draws a sample from the cup, performs analysis and transmits results. Clinician or patient then transfers cup for downstream testing. The device may be mobile and portable with a battery backup. The motor, instruction screen, scanners, speakers, urine collection mechanism, and urine analysis subsystem may all be powered using the battery. The battery can be rechargeable by removing the battery and plugging it into a charger that is plugged into a power outlet. The battery can additionally or alternatively be wirelessly charged.

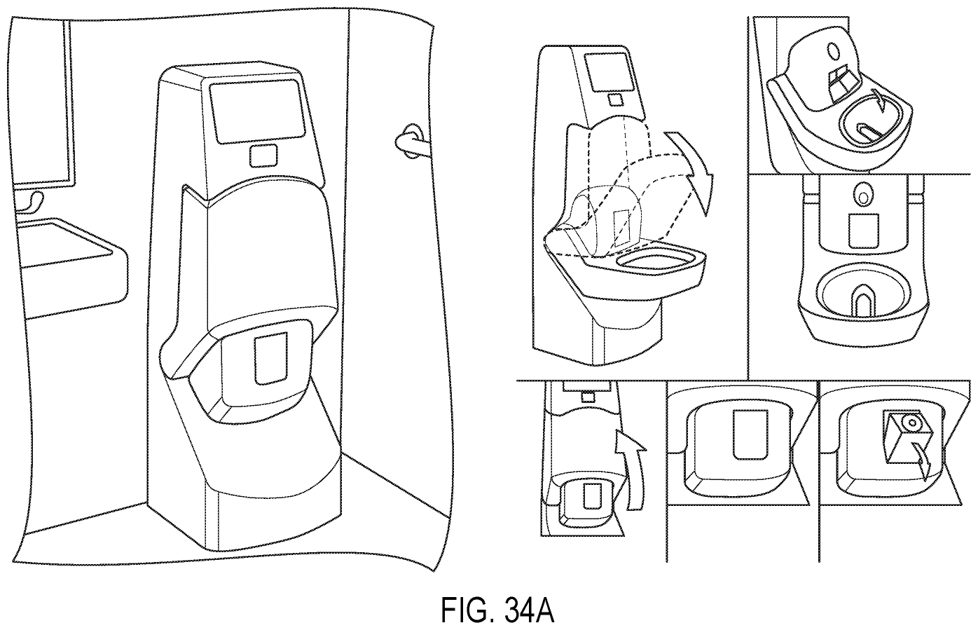

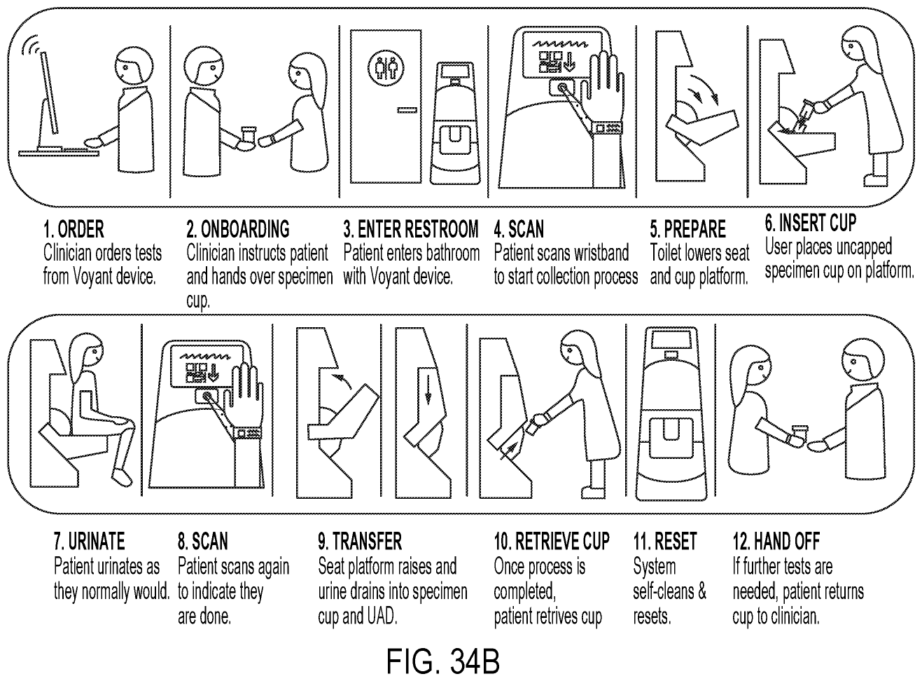

Referring now to FIG. 34, another example of the device is shown. The device may be a standalone system with integrated collection.

Referring now to FIGS. 34 and 34A, the process flow for using the stand-alone device will be discussed. The clinician orders tests from the device. The clinician then instructs the patient and hands over the urine collector 7. The patient enters the bathroom with the device. The patient scans their barcode to start the process. The device lowers the platform. The patient inserts the cup 8 as instructed. The patient urinates as normal. The patient scans the barcode again to indicate they are done. The urine is transferred to the device and the urine collector 7. The patient retrieves the urine collector 7. The system resets itself as it analyzes the urine. The patient hands off the sample to the clinician.

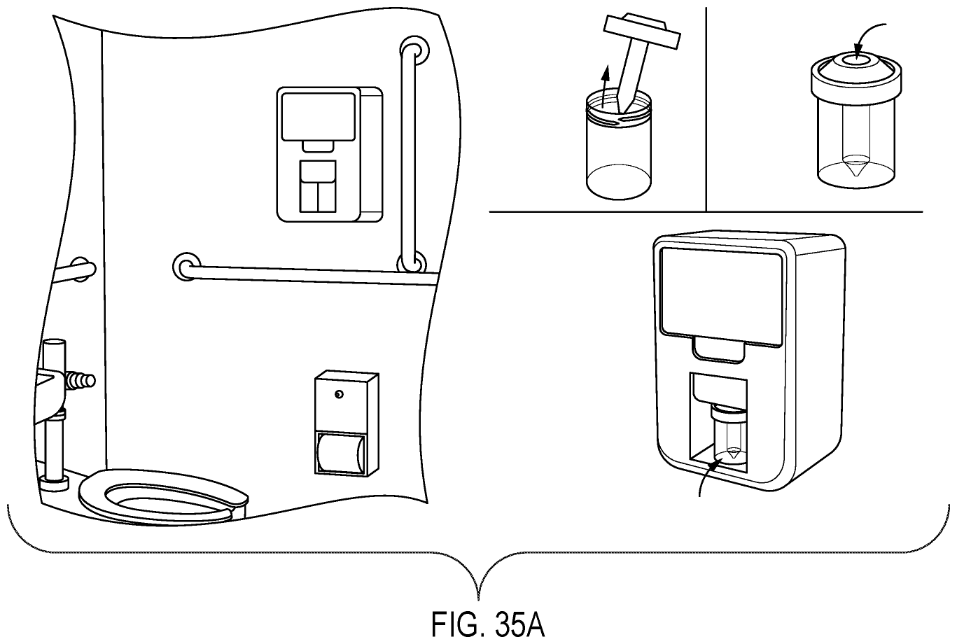

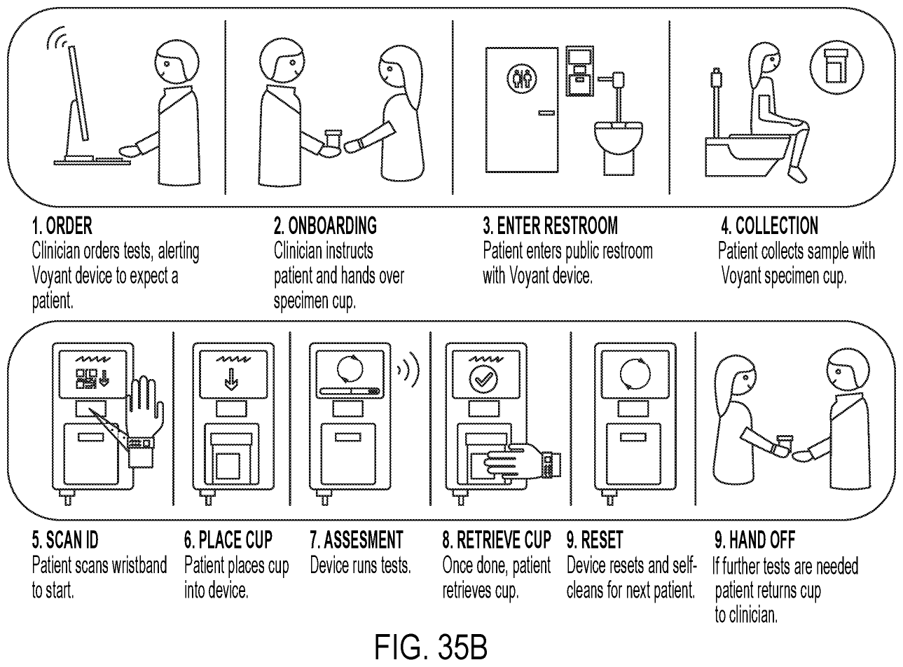

Referring now to FIG. 35, the device without the urine collection mechanism will be discussed. The urine analysis device may be mounted on the wall and collect urine from a specially-designed cup 8.

Referring now to FIGS. 35 and 35A, the process flow for using the device without urine collection will be discussed. The clinical orders tests, alerting the device to expect a patient. The clinician instructs the patient and hands over the specimen cup 8. The patient enters the restroom with the device. The patient collects the sample in the urine cup 8. The patient scans the barcode to start the process. The patient places the cup 8 in the device. The device runs the tests. Once the device analysis is complete, the patient retrieves the cup 8. The device resets itself for the next patient. The patient hands off the sample to the clinician.

Another method for the installation of the urine collector will be discussed. The urine collector may be installed inside the bowl of the toilet and connect to the plumbing of the toilet.

Another set of tests that may be tested by the diagnostic device will be discussed. The diagnostic device could test for pH, chloride levels, glucose, lactate, sodium, potassium, and other metabolites, hormones, and proteins that could be in urine. The diagnostic device could use chemistry-based tests, antibody-based tests, or aptamer-based tests for diagnosis.

The device may have a custom flush valve that replaces the original flush valve of the toilet. The custom flush valve may route water to the toilet and to the device.

An add-on device to toilets as described herein may automate point-of-care pregnancy urine testing from specimen collection through testing to delivery of results, improving operational efficiencies in EDs. To satisfy complementary clinical and operational requirements, the device also collects and transfers sterile midstream urine to an external urine collection cup for additional point-of-care (POC) and centralized laboratory testing. Against available options in manual and semi-automated POC tests, the device described herein may provide better or comparable performance in terms of speed, cost, accuracy, and user experience. In the ED, the solution will decrease patient length of stay, reduce labor costs, reduce human errors, and improve the patient and clinical staff experience. The embodiments described herein will improve the quality, safety, and efficiency of ED care.

The existing manual and semi-automated POC pregnancy testing systems were analyzed and it was determined that the existing systems did not overcome and satisfy these 5 key technical challenges and requirements needed for a POC system to be considered a diagnostic system that fully automates POC urine pregnancy testing: 1) Full automation from urine collection through testing to delivery of results; 2) Sterile collection and transfer of midstream urine without dilution; 3) Control of sample volume; 4) Test strip automation; 5) Prevention of cross-contamination of pregnancy test results from different patients.

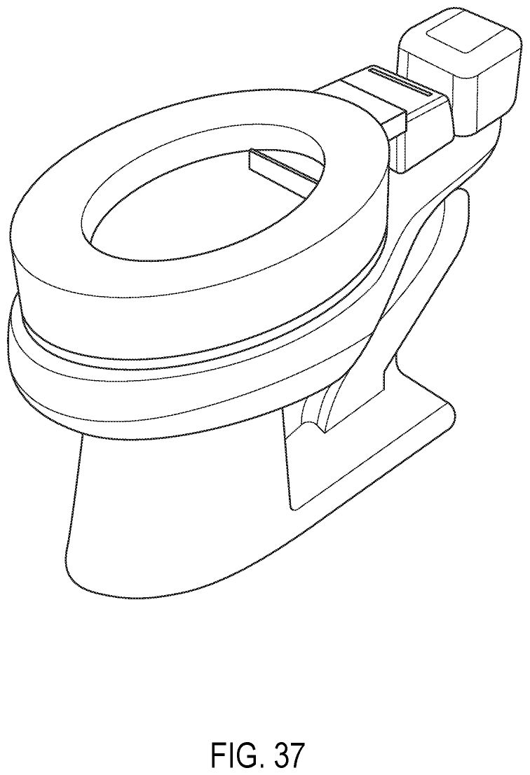

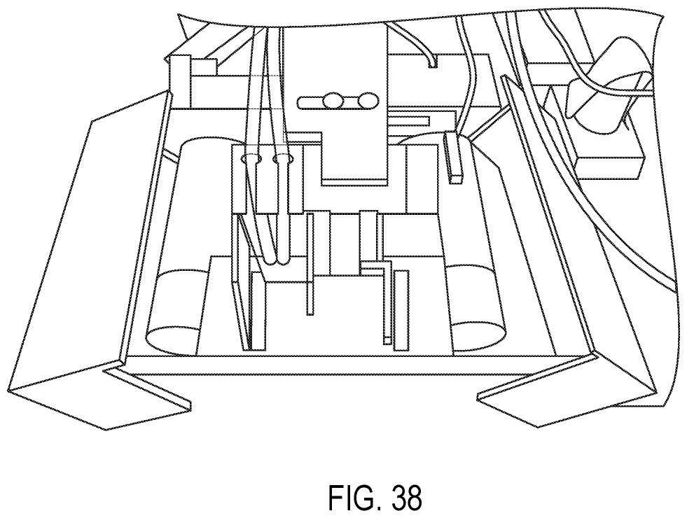

Referring now to FIGS. 36-39, the system described herein was designed with the features described below to overcome these challenges. The system was designed with two main sub-systems: 1) the urine collector 7 and 2) the diagnostic device 6. The urine collection sub-system (urine collector) 7 is deployed over the toilet bowl and collects sterile midstream urine from the patient without dilution of urine. The diagnostic device 6 is mounted on top of the back end of the toilet against the wall. The urine collector transfers the collected sterile midstream urine into the diagnostic device 6 for hCG analysis, and into an external urine collector 7 for additional POC (general urinalysis) or centralized laboratory testing (urine culture).

Referring now to FIGS. 36-39, the full automation from urine collection through testing to delivery of results will be discussed. The patient simply walks into the restroom, places her urine collector 7 on the location 10 while the urine collector 7 is in its undeployed Up state. The patient scans her hospital barcode ID wristband on the device's barcode scanner to identify herself and send a pregnancy test order to the device. Scanning the wristband activates the urine collector's Down state, triggering the urine collector 7 to automatically pivot onto and deploy over the toilet bowl through a motorized hinge.

Referring now to FIGS. 36-39, the full automation from urine collection through testing to deliver of results will continue to be discussed. From there, the patient urinates into the toilet bowl as she normally would into a regular toilet. The urine collector's absorbant-coated, film-covered central platform 2 collects the sterile midstream urine without dilution before the urine reaches the toilet bowl. When the patient is finished urinating, she will scan her barcode ID wristband again to indicate completion. The urine collector 7 will return to its Up state to transfer via gravity a portion of the urine into the diagnostic device 6 where the urine is processed and analyzed for hCG. Simultaneously, the urine collector 7 transfers via gravity a portion of the urine into the external urine collector 7. The patient picks up the urine collector 7 and leaves the restroom as the diagnostic device 6 completes its hCG analysis. The patient leaves the urine collector 7 at a location designated by clinical staff inside or outside of the restroom. The urine platform 2 rolls away the used film 18 that covers the central platform, and rolls out fresh film that covers the central platform 2 to make the platform 2 ready to collect the next patient's midstream sterile urine.

Referring to FIGS. 35-39, the diagnostic device 6 will be discussed. Parallel automated processes occur within the diagnostic device 6 when it receives the urine. The diagnostic device 6 transports and processes the urine via tubing and a peristaltic pump 21 through a sample volume control and dispense mechanism 22 to drop a fixed volume of urine onto a test strip. Excess urine that is not needed for the analysis is transported back to the toilet bowl via tubing and a peristaltic pump. A reel-to-reel, test strip automation mechanism 23 brings a test strip 24 underneath the sample volume control and dispense mechanism 22 to receive the urine sample. Then, the test strip automation mechanism brings the test strip underneath an optical imager, where the test strip is imaged 3 minutes after receiving the urine sample. In parallel, peristaltic pumps 21 wash the pregnancy analyzer with a wash buffer and water to denature and remove residual hCG from the pregnancy analyzer to prevent cross-contamination of pregnancy test results from different patients.

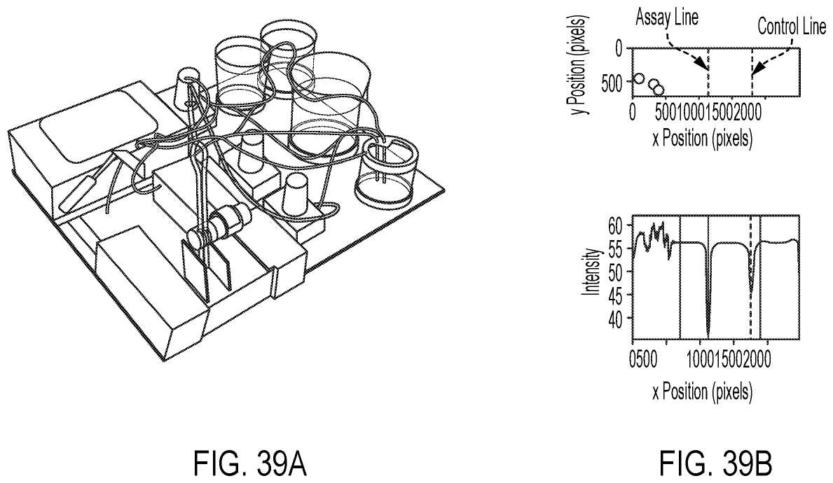

Referring to FIG. 39, the image processing algorithm will be discussed. An image processing algorithm processes the test strip image to determine the qualitative pregnancy status of the patient (hCG positive (pregnant) or hCG negative (not pregnant)). The system sends this test result to the ED's electronic health record system through Wi-Fi. Finally, the system flushes the toilet. Altogether, the device's automated process takes 5 minutes from collection of urine through testing to delivery of results. A microprocessor circuit 30 controls the entire system for the whole process.

Referring to FIGS. 36-39, the sterile collection and transfer of midstream urine without dilution will be discussed. The urine collector 7 is hinged onto the toilet behind the toilet bowl. By default, the urine collector 7 is in its Up state away from the toilet bowl, so as to not interfere with non-testing urination, defecation, and other general usage of the toilet by people. The hinge is motorized, so when the patient scans her barcode ID wristband, the hinge actuates and rotates the urine collector 7 downward onto the toilet seat, into its Down state.

The patient sits on or squats above the urine collector 7 as if it were a toilet seat, and urinates into the collector 7 as if it were the toilet bowl. The urine collector 7, made from polyoxymethylene plastic (DELRIN), has a central platform 2 above the toilet bowl that is rigid and curved. This platform 2 spans the toilet bowl, so the patient may urinate into the platform without aiming, and the platform's curved basin will collect the urine. It is understood that the central platform may be made from other plastics as well (e.g., polyvinyl chloride (PVC), polypropylene (PP), polyethylene (PE), polystyrene (PS), nylon, polyethylene terephthalate (PET), polyimide (PA), polycarbonate (PC), acrylonitrile butadiene (ABS), polyetheretherketone (PEEK), and/or polyurethane (PU)). In some embodiments, the central platform 2 may be made from metal (e.g., stainless steel and/or aluminum). The central platform 2 may be attached to the overall device or the diagnostic device 6. The diagnostic device 6 may be attached to the flushometer of the toilet. In some embodiments, the central platform 2 may be attached to the toilet seat hinge above the toilet bowl.

The central platform has an array of holes that are connected to a small, membrane, vacuum pump (e.g., a direct current (DC) diaphragm vacuum pump) situated inside of the platform. For each patient, the platform is covered by fresh unused, water-insoluble polyethylene terephthalate (PET) plastic film, which is rolled onto the platform by a reel-to-reel, stepper motor mechanism and a guide wire on the urine collection sub-system. The vacuum pump generates a vacuum that holds the fresh film down against the platform, causing the film to conform to the curved shape of the platform 2. When the patient urinates into the urine collector 7, the urine is collected by the fresh film over the platform, maintaining the sterility of the urine sample since urine from different patients never touch the same surface. Moreover, since the platform collects the urine without the urine reaching the toilet bowl where water is, the sample is not diluted. When the urine collector 7 pivots upward to transfer the urine into the pregnancy analyzer and external urine collection cup, sterility is also maintained because the film covers the rigid contoured base 14 and holes beneath the guide wire.

The filmis coated with sodium polyacrylate, which swells upon contact with the initial urine and absorbs the initial urine, so that only the midstream urine and onward is collected and transferred by the urine collector 7.

The sample volume control and dispense mechanism will now be discussed. The sample volume control and dispense mechanism 22 collects a fixed volume of urine that is dispensed onto each test strip. This mechanism is a y-shaped tubing apparatus controlled by a three-way solenoid valve. The tubing pathway bifurcates into two tubing pathways. The first pathway collects a fixed volume of urine to be dropped onto each test strip. The second pathway allows excess urine not needed for the analysis to flow through into a waste basin, where the urine is routed into the toilet bowl to be flushed. The sample volume may be a fixed volume (e.g., 1 mL of urine), which would be the same amount for all patients. Alternatively, the sample volume may depend on the conditions being tested. In some embodiments, the 3-way solenoid may be controlled by an open/close valve that is electronically powered. The first pathway may be filled by closing the waste pathway with the valve to allow the first pathway to fill with the fixed volume (e.g., 1 mL), and then the waste (second) pathway may open for additional waste urine while closing the first pathway. After a set amount of time (e.g., 30 seconds), the first pathway may again be opened (and the second and waste pathway may again be closed by the valve) to drop the fixed volume (e.g., 1 mL) of urine onto the test strip. The waste basin may be shaped like a rectangular prism with an open ceiling and a hole for a pipe at the bottom. Alternatively, the waste basin may be circular or pyramidal. The excess urine goes first into the waste basin (e.g., because, in some cases, the urine analysis subsystem may be sufficiently separate from the toilet bowl to allow for the waste to go directly to the bowl).

The test strip automation will now be discussed. A disposable cassette holds a multitude of lateral flow immunochromatographic assay urine pregnancy test strips 24 on a film in a test strip automation mechanism 23 (e.g., in a reel-to-reel configuration). The clinical staff inserts the cassette into the pregnancy analyzer's reel-to-reel, stepper motor mechanism, which automates the usage of test strips for the next 100 patients. For each patient, the stepper motors spin to bring a new test strip under the sample volume control and dispense mechanism to receive the urine sample, and then under the optical imager for analysis. The dynamic positioning of the test strip is accurately controlled by opto-interrupter sensors.

Prevention of cross-contamination of pregnancy test results from different patients will now be discussed. During each analysis cycle, the pregnancy analyzer washes itself with a diluted sodium hypochlorite (bleach) solution, which denatures and removes any residual hCG that is on tubing walls or urine collection basins without damaging the tubing or basins. The diagnostic device 6 then washes away the sodium hypochlorite with water so that hCG results at sensitivity are not impacted by residual sodium hypochlorite.

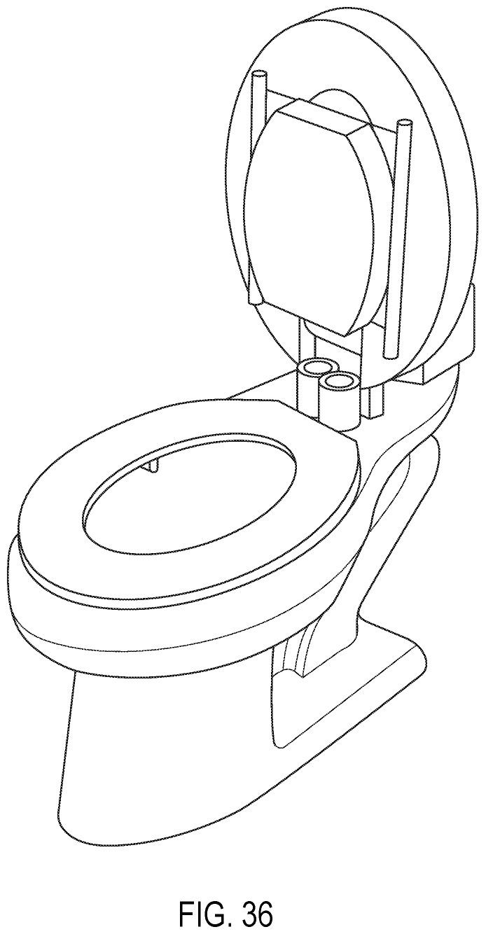

FIGS. 36 and 37 illustrate the design of the second-generation urine collector 7. After the urine collector receives the patient's urine, the motorized hinge actuates to rotate the sub-system upward back into its Up state away from the toilet bowl. Gravity causes the collected midstream urine to drain and collect into the rigid contoured base 14. The rigid contoured base 14 is covered with PET plastic film to maintain sterility. This plastic film contains two smaller circular layers of water-soluble, polyvinyl alcohol film (PVA) that the urine collects over, which dissolve a few seconds later. This prevents urine from prematurely draining out of the collector 7 before the collector has repositioned itself in its Up state. Periodically, the film may have two holes/dissolvable stoppers. For example, in each disposable film cartridge, there could be 100 sets of two holes/dissolvable stoppers. The dissolving PVA film may be adhered over each hole/dissolvable stopper. These PVA films may be between 50 mm-100 mm in thickness, in some embodiments. Further, each PVA film over each hole may have multiple layers. The film may be in a reel-to-reel cartridge. Additionally, the diameter of the circular PVA films could be between 0.5 inches and 2 inches, in some embodiments.

When the PVA layers dissolve, gravity will cause the urine to drain into both the pregnancy analyzer's urine collection basin and the external urine collection cup. The pregnancy analyzer analyzes the urine for the pregnancy status of the patient. Meanwhile, the patient removes the external urine collection cup and leaves it at a location designated by the clinical staff inside/outside of the restroom.

When all of the urine has drained from the urine collector, a solenoid valve releases the vacuum generated by the vacuum pump from the urine collector, releasing the PET film from the central platform 2. The reel-to-reel stepper motors roll away used film and bring unused film over the central platform. The vacuum reactivates to hold the fresh film down against the central platform, ready to collect the next patient's sterile midstream urine.

The PET film is on a roll within a disposable cassette that is discarded/replaced by clinical staff once per day. The PET film is coated with a layer of sodium polyacrylate (SPA). SPA swells upon contact with the initial urine stream and absorbs it. SPA saturates and does not absorb the sterile midstream urine. Hence, the urine collector's PET film only collects/transfers sterile midstream urine into the external urine collection cup and the pregnancy analyzer.

The materials and components will now be discussed. The materials and components described that comprise the urine collector are commonly used in prototyping due to their ease of prototyping, or easily sourced from off the shelf.

Conforming the film to the curved shape of the central platform 2 via the vacuum is a potential technical challenge. To mitigate this risk, the PET film will be looped around a guide wire at the top of the drainage basin to guide its positioning, and opto-interrupter sensors will be used to accurately position the PET film. If needed for optimization, alternative common film materials may be used such as polyethylene or polyvinylidene chloride.

A use case for the invention will now be discussed. A diagnostic smart toilet platform device according to example embodiments may be deployed on toilets in the home and in clinical settings, such as dialysis clinics, to improve chronic kidney disease management and prevent patients from “crashing” into dialysis. For Stage 1 to 3 chronic kidney disease patients (early to mid-stage patients) and Stage 4 to 5 chronic kidney disease patients (late stage patients), devices disclosed herein may monitor the concentration of urinary chronic kidney disease biomarkers, such as beta-trace protein, in the patients whenever the patients urinate into toilets.

This monitoring may take place at various frequencies, such as whenever the patients urinate into a toilet, multiple times per day, once per day, once per week, multiple times per week, once per month, multiple times per month, multiple times per year, once per year, etc.

By monitoring the urinary biomarker concentrations, clinicians, such as nephrologists, may remotely monitor the progression of the chronic kidney disease in patients over time, especially from the patient's home. By monitoring the progression of chronic kidney disease, the clinician may manage the chronic kidney disease, and plan with the patient in advance what the appropriate treatment options are, preventing the patient from “crashing” into dialysis when the patient needs emergency dialysis because the chronic kidney disease has not been monitored, managed, or planned for treatment in advance by the clinician with the patient. When the patient “crashes” into dialysis, clinical outcomes decrease, economic costs increases, and the patient's lifestyle, lifestyle flexibility, and patient experience are adversely impacted. The optimal dialysis treatment option is not able to be performed without the pre-planning.

As an example, when an example device is installed in the patient's home, the clinician may remotely monitor when a Stage 3 patient is approaching Stage 4 of chronic kidney disease, when patients typically need to begin dialysis. This provides the clinician and patient enough time to plan the appropriate treatment options for the late stage chronic kidney disease (Stage 4 or Stage 5), such as whether the patient should be prescribed peritoneal dialysis from the home or receive hemodialysis in a dialysis clinic. A similar application may be performed for Stage 4 patients approaching Stage 5.

To receive peritoneal dialysis from the home, the patients need to be trained and the clinicians require advance preparatory time, which can take at least 2 weeks. Currently, when a patient “crashes” into dialysis, pre-planning of the treatment has not occurred, so there is not enough advanced preparatory or training time to enable the patient to be put on peritoneal dialysis from the home, which can improve clinical outcomes, reduce economic costs, and improve the patient's lifestyle, lifestyle flexibility, and patient experience. As a result, the patient has to receive emergency hemodialysis from the hospital or a dialysis clinic.

Example devices disclosed herein may convert the urinary biomarker concentrations (such as beta-trace protein) into the glomerular filtration rate, which is used to determine the stage and progression of the chronic kidney disease.

A diagnostic smart toilet platform device according to example embodiments may test the urine of chronic kidney disease patients to diagnose and monitor them for early to late stages of chronic kidney disease (Stages 1 through 5), from the home or in clinical settings. To achieve this goal, the device may test the urine for multiple threshold, beneath-threshold, between-threshold, and above-threshold concentrations of urinary biomarkers of chronic kidney disease. Urinary biomarkers of chronic kidney disease may include:

i. Albumin

ii. Creatinine

iii. Albumin to creatinine ratio

iv. Cystatin C

v. Cystatin C to creatine ratio

vi. β2-microglobulin

vii. β2-microglobulin to creatinine

viii. Retinol-binding protein (RBP)

ix. Retinol-binding protein (RBP) to creatinine ratio

x. Beta-trace protein

xi. Beta-trace protein to creatinine ratio.

Other urinary biomarkers of chronic kidney disease are also possible. Devices disclosed herein may test for one or more urinary biomarkers of chronic kidney disease using test strips packaged within one or more test cartridges. These test strips target the one or more urinary biomarkers of chronic kidney disease. The test strips may be lateral flow test strips that are antibody or aptamer based. The test strips may also be based on general chemistries.

A diagnostic smart toilet platform device according to example embodiments may test the urine of chronic kidney disease patients to diagnose and monitor them for uremic toxins (and/or biomarkers) associated with cardiovascular disease or chronic kidney disease, from the home or in clinical settings. To achieve this goal, the device may test the urine for multiple threshold, beneath-threshold, between-threshold, and above-threshold concentrations of the uremic toxins or biomarkers associated with cardiovascular disease or chronic kidney disease. In some embodiments, the device may test for the uremic toxins or biomarkers qualitatively or determine their concentrations semi-quantitatively or quantitatively. The concentrations of the uremic toxins or biomarkers may be determined by an onboard controller in the device. The thresholds may be predefined, in some embodiments. Further the device may be configured to determine a ratio of multiple biomarkers or uremic toxins by comparing their respective concentrations. For example, the device may be configured to determine a concentration of urea and a concentration of creatinine and then, based on the two concentrations, calculate a urea to creatinine ratio. This could similarly be done for other biomarkers and/or uremic toxins, as well. Uremic toxics or biomarkers associated with cardiovascular disease or chronic kidney disease may include:

i. Urea

ii. Urea to creatinine ratio

iii. Phosphate

iv. Phosphate to creatinine ratio

v. Creatinine

vi. Parathyroid hormone (PTH)

vii. Parathyroid hormone to creatinine ratio

viii. Beta 2 microglobulin

ix. Beta 2 microglobulin to creatinine ratio

x. Cystatin C

xi. Cystatin C to creatinine ratio

xii. Myoglobin

xiii. Myoglobin to creatinine ratio

xiv. Kappa free light chains

xv. Kappa free light chains to creatinine ratio

xvi. Complement factor D

xvii. Complement factor D to creatinine ratio

xviii. Interleukin-6

xix. Interleukin-6 to creatinine ratio

xx. Alpha 1 microglobulin

xxi. Alpha 1 microglobulin to creatinine ratio

xxii. YKL-40

xxiii. YKL-40 to creatinine ratio

xxiv. Lambda free light chains

xxv. Lambda free light chains to creatinine ratio

xxvi. Albumin

xxvii. Albumin to creatinine ratio

xxviii. Indoxyl sulfate

xxix. Indoxyl sulfate to creatinine ratio

xxx. Indoxyl glucuronide

xxxi. Indoxyl glucuronide to creatinine ratio

xxxii. Indoleacetic acid

xxxiii. Indoleacetic acid to creatinine ratio

xxxiv. P-Cresyl sulfate

xxxv. P-Cresyl sulfate to creatinine ratio

xxxvi. P-Cresyl glucuronide

xxxvii. P-Cresyl glucuronide to creatinine ratio

xxxviii. Phenyl sulfate

xxxix. Phenyl sulfate to creatinine ratio

xl. Phenyl glucuronide

xli. Phenyl glucuronide to creatinine ratio

xlii. Phenylacetic acid

xliii. Phenylacetic acid to creatinine ratio

xliv. Phenylacethyl glutamine

xlv. Phenylacethyl glutamine to creatinine ratio

xlvi. Hippuric acid

xlvii. Hippuric acid to creatinine ratio

xlviii. 4-Ethylphenyl sulfate

xlix. 4-Ethylphenyl sulfate to creatinine ratio

l. 3-Carboxy-4-methyl-5-propyl-2-furanpropionic acid

li. 3-Carboxy-4-methyl-5-propyl-2-furanpropionic acid to creatinine ratio

Other uremic toxins associated with cardiovascular disease or chronic kidney disease are also possible. Devices disclosed herein may test for one or more uremic toxins or biomarkers associated with cardiovascular disease or chronic kidney disease using test strips packaged within one or more test cartridges. These test strips target the one or more uremic toxins or biomarkers associated with cardiovascular disease or chronic kidney disease. The test strips may be lateral flow test strips that are antibody or aptamer based. The test strips may also be based on general chemistries.

Devices disclosed herein may analyze the effluent of patients from dialysis machines for multiple threshold, beneath-threshold, between threshold, and above-threshold concentrations of biomarkers and/or uremic toxins associated with cardiovascular disease or chronic kidney disease. The effluent waste tubing line from a dialysis machine may be extended and routed to above the urine collection sub-system of the device on the toilet described herein, for example. Further, the effluent during dialysis may drain through the waste tubing line from the dialysis machine over the urine collection sub-system. The device may then collect the effluent in the urine collection sub-system and transport the effluent to the analysis sub-system to analyze the effluent. The device described herein may test the effluent with the same or similar process used to test the urine. The biomarkers or uremic toxins associated with cardiovascular disease or chronic kidney disease may include:

i. Albumin

ii. Creatinine

iii. Albumin to creatinine ratio

iv. Cystatin C

v. Cystatin C to creatine ratio

vi. β2-microglobulin

vii. β2-microglobulin to creatinine

viii. Retinol-binding protein (RBP)

ix. Retinol-binding protein (RBP) to creatinine ratio

x. Beta-trace protein

xi. Beta-trace protein to creatinine ratio.

xii. Urea

xiii. Urea to creatinine ratio

xiv. Phosphate

xv. Phosphate to creatinine ratio

xvi. Creatinine

xvii. Parathyroid hormone (PTH)

xviii. Parathyroid hormone to creatinine ratio

xix. Beta 2 microglobulin

xx. Beta 2 microglobulin to creatinine ratio

xxi. Cystatin C

xxii. Cystatin C to creatinine ratio

xxiii. Myoglobin

xxiv. Myoglobin to creatinine ratio

xxv. Kappa free light chains

xxvi. Kappa free light chains to creatinine ratio

xxvii. Complement factor D

xxviii. Complement factor D to creatinine ratio

xxix. Interleukin-6

xxx. Interleukin-6 to creatinine ratio

xxxi. Alpha 1 microglobulin

xxxii. Alpha 1 microglobulin to creatinine ratio

xxxiii. YKL-40

xxxiv. YKL-40 to creatinine ratio

xxxv. Lambda free light chains

xxxvi. Lambda free light chains to creatinine ratio

xxxvii. Albumin

xxxviii. Albumin to creatinine ratio Indoxyl sulfate

xxxix. Indoxyl sulfate to creatinine ratio

xl. Indoxyl glucuronide

xli. Indoxyl glucuronide to creatinine ratio

xlii. Indoleacetic acid

xliii. Indoleacetic acid to creatinine ratio

xliv. P-Cresyl sulfate

xlv. P-Cresyl sulfate to creatinine ratio

xlvi. P-Cresyl glucuronide

xlvii. P-Cresyl glucuronide to creatinine ratio

xlviii. Phenyl sulfate

xlix. Phenyl sulfate to creatinine ratio

l. Phenyl glucuronide

li. Phenyl glucuronide to creatinine ratio

lii. Phenylacetic acid

liii. Phenylacetic acid to creatinine ratio

liv. Phenylacethyl glutamine

lv. Phenylacethyl glutamine to creatinine ratio

lvi. Hippuric acid

lvii. Hippuric acid to creatinine ratio

lviii. 4-Ethylphenyl sulfate

lix. 4-Ethylphenyl sulfate to creatinine ratio

lx. 3-Carboxy-4-methyl-5-propyl-2-furanpropionic acid

lxi. 3-Carboxy-4-methyl-5-propyl-2-furanpropionic acid to creatinine ratio

Other biomarkers or uremic toxins associated with cardiovascular disease or chronic kidney disease are also possible. Devices disclosed herein may test for one or more biomarkers or uremic toxins associated with cardiovascular disease or chronic kidney disease using test strips packaged within one or more test cartridges. These test strips target the one or more biomarkers or uremic toxins associated with cardiovascular disease or chronic kidney disease. The test strips may be lateral flow test strips that are antibody or aptamer based. The test strips may also be based on general chemistries.

The urine collection mechanism of a smart toilet platform device according to example embodiments may be attached to an effluent line from dialysis machines, such as an automated peritoneal dialysis instrument in the home or clinical setting, or a hemodialysis instrument in the clinical setting or home. The urine collection mechanism of a device according to example embodiments will collect the patient's effluent coming out of the effluent line, and send the effluent into the urine analysis sub-system of the device to analyze the effluent for threshold concentrations of biomarkers associated with chronic kidney disease or cardiovascular disease and/or threshold concentrations of uremic toxins associated with chronic kidney disease or cardiovascular disease.

Alternatively, the urine analysis sub-system of a device according to example embodiments may be in an off-the-toilet architectural configuration and integrated with or by the dialysis machine to test the effluent in the effluent line at or near the dialysis machine.

For chronic kidney disease and affiliated co-morbidities such as cardiovascular disease, urinary biomarker concentration data, effluent biomarker concentration data, uremic toxin concentration data from the urine or effluent, may be sent by the device into a cloud-based server that clinicians can access. The clinician may use this diagnostic data to remotely or non-remotely optimize the dialysis treatment prescriptions (or other types of treatments) of their chronic kidney disease patients in the home or clinic. In particular, for at-home, automated peritoneal dialysis, some treatment prescription parameters that the clinician may remotely modify include how much fluid volume is sent into the patient, how much fluid volume is pulled out of the patient, number of treatment cycles, or types of fluids.

Devices disclosed herein may be integrated with a toilet seat that is embedded with sensors for monitoring cardiovascular diseases and heart failure, to collectively monitor for co-morbid chronic conditions, such as chronic kidney disease, diabetes, and/or cardiovascular disease. The toilet seat sensors monitor heart rate, blood pressure, blood oxygenation levels, and the patient's weight and stroke volume. The sensors in the toilet seat may allow for an electrocardiogram, photoplethysmogram, and/or ballistocardiogram.

Devices disclosed may be attached to a power source that may be re-charged remotely. The remote re-charge may be triggered when the power source runs out of power or when the power-source reaches a threshold of power. The trigger may be performed automatically or manually by a medical staff. The remote connection to the power source may be enabled by wireless connectivity, Bluetooth connectivity, or other non-wired connectivity.

Further, the remotely re-charged power source may be made small enough to fit within the device as opposed to act as an external power source to the device. The power source may be remotely turned off when the device is not in use.

The device may collect anonymized and Health Insurance Portability and Accountability Act (HIPAA)-compliant data of patients. Data of patients may be stored internally or externally (e.g., on a non-transitory, computer-readable medium, such as a hard drive). The data may be incorporated into machine-learning algorithms to predict disease progression and impact of treatment. In particular, monitoring data may be collected to perform predictive diagnostics for urinary tract infections, particularly, but not limited to, diagnosing urinary tract infections in senior-care facilities.

1. A device comprising:
an initial collection component configured to collect a midstream urine sample from a patient;
a plurality of test strips configured to indicate a condition of the patient when exposed to the midstream urine sample;
a fluid transportation system configured to:
transport a portion of the midstream urine sample from the initial collection component to at least one test strip of the plurality of test strips; and
expose the at least one test strip to the portion of the midstream urine sample;
a sensor configured to capture an image of the at least one test strip exposed to the portion of the midstream urine sample, wherein the image of the at least one test strip indicates the condition of the patient at a point of care;
a computing device configured to analyze the image of the at least one test strip captured by the sensor in order to determine the condition of the patient at the point of care;
a motor configured to position the at least one test strip near the sensor after the at least one test strip is exposed to the portion of the midstream urine sample; and
an additional collection component configured to collect an additional portion of the midstream urine sample from the initial collection component for central laboratory testing.

2. The device of claim 1,
further comprising a midstream urine collection seat,
wherein the midstream urine collection seat comprises the initial collection component,
wherein the seat is raised or lowered mechanically using an electric motor,
wherein the seat is mechanically lowered into a lowered position to collect urine from the patient for testing, and
wherein the seat is mechanically raised into a raised position after urine has been collected from the patient.

3. The device of claim 1,
wherein the additional collection component comprises a sterile collection cup,
wherein the sterile collection cup has one or more barcode stickers disposed thereon, and
wherein a barcode on the one or more barcode stickers matches a barcode on a bracelet worn by the patient.

4. The device of claim 1,
wherein the initial collection component comprises a rigid central platform with a film overlaid over the platform to absorb an initial urine stream and collect the midstream urine sample,
wherein:
the rigid central platform comprises an anti-microbial coating and a hydrophobic coating;
the film comprises a trough in a center of the film;
the film comprises absorbent strips spaced at discrete intervals on the film to absorb the initial urine stream and collect the midstream urine sample, wherein the absorbent strips comprise sodium polyacrylate;
the film comprises holes; or
the film comprises shapes made of water-soluble plastic,
wherein the film is stabilized using a guide wire,
wherein the film comprises two circular layers of water-soluble, polyvinyl alcohol (PVA) over which the midstream urine sample collects, and
wherein the midstream urine sample dissolves the two circular layers within a few seconds of collecting over the two circular layers.

5. The device of claim 1,
wherein the initial collection component comprises a rigid central platform made of polyoxymethylene plastic that is coated with an anti-microbial coating and a hydrophobic coating,
wherein the initial collection component comprises a rigid central platform comprising an array of holes connected to a membrane and a vacuum pump located inside the rigid central platform, and
wherein the initial collection component comprises a film and a rigid contoured base.

6. The device of claim 1, wherein the initial collection component comprises a backside curvature configured to hold the midstream urine sample when the initial collection component is raised.

7. The device of claim 1,
wherein the initial collection component comprises a film, and
wherein, after use, the film is unrolled from over the initial collection component and a replacement film is rolled over the initial collection component.

8. The device of claim 1, further comprising an instruction screen configured to provide a demonstration on how to use the device, wherein the demonstration comprises static drawings, videos, or audio.

9. The device of claim 1, wherein the initial collection component comprises a platform having an open space defined therein for disposal of toilet paper into a toilet bowl below the platform.

10. The device of claim 1,
wherein the additional collection component comprises a sterile collection cup,
wherein the device further comprises a predefined location for the additional collection component, and
wherein the predefined location is revealed using an electronic signal or mechanical method.

11. The device of claim 1,
wherein the initial collection component comprises a rigid platform overlaid with film,
wherein the platform is positioned over a toilet bowl,
wherein the additional collection component comprises a sterile collection cup,
wherein the platform is raised in response to a flush lever of the toilet being pushed, and
wherein the midstream urine sample is at least partially transferred from the collection component to the additional collection component upon the platform being raised.

12. The device of claim 1, further comprising a refillable reservoir configured to provide clean water for flushing and device sterilization functions.

13. The device of claim 1, further comprising a battery backup, wherein the device is mobile and portable.

14. The device of claim 1,
wherein transporting the portion of the midstream urine sample from the initial collection component to the at least one test strip of the plurality of test strips is performed using a y-shaped tubing apparatus controlled by a three-way solenoid valve and a tubing pathway,
wherein the tubing pathway bifurcates into a first tubing pathway and a second tubing pathway,
wherein the first tubing pathway is directed to the at least one test strip, and
wherein the second tubing pathway is directed to a waste basin in the device and then to a toilet bowl.

15. The device of claim 1,
wherein the at least one test strip is configured to indicate the presence of a uremic toxin, a biomarker associated with cardiovascular disease, or a biomarker associated with chronic kidney disease, and
wherein the uremic toxin, the biomarker associated with cardiovascular disease, or the biomarker associated with chronic kidney disease comprise urea, phosphate, creatinine, parathyroid hormone (PTH), beta 2 microglobulin, cystatin C, myoglobin, kappa free light chains, complement factor D, interleukin-6, alpha 1 microglobulin, YKL-40, lambda free light chains, albumin, indoxyl sulfate, indoxyl glucuronide, indoleacetic acid, P-Cresyl sulfate, P-Cresyl glucuronide, phenyl sulfate, phenyl glucuronide, phenylacetic acid, phenylacethyl glutamine, hippuric acid, 4-Ethylphenyl sulfate, or 3-Carboxy-4-methyl-5-propyl-2-furanpropionic acid.

16. The device of claim 1, further comprising:
an additional sensor configured to capture an image of the midstream urine sample,
wherein the image of the midstream urine sample indicates an extent of dehydration of the patient, and
wherein the computing device is configured to analyze the image of the midstream urine sample in order to determine the extent of dehydration of the patient by determining a color and darkness of the midstream urine sample; and
a refractometer,
wherein the at least one test strip indicates the extent of dehydration of the patient, and
wherein the computing device is configured to analyze the image of the at least one test strip or data from the refractometer in order to determine the extent of dehydration of the patient by determining a concentration of the midstream urine sample or a specific gravity of the midstream urine sample.

17. The device of claim 1,
wherein the condition of the patient comprises ovulation, and
wherein the plurality of test strips are configured to indicate the presence of luteinizing hormone.

18. The device of claim 1, wherein the device is configured to fit around or over consumer toilets in an at-home setting or a senior care setting.

19. A system comprising:
a device comprising:
an initial collection component configured to collect a midstream urine sample from a patient;
a plurality of test strips configured to indicate a condition of the patient when exposed to the midstream urine sample;
a fluid transportation system configured to:
transport a portion of the midstream urine sample from the initial collection component to at least one test strip of the plurality of test strips; and
expose the at least one test strip to the portion of the midstream urine sample;
a sensor configured to capture an image of the at least one test strip exposed to the portion of the midstream urine sample, wherein the image of the at least one test strip indicates the condition of the patient at a point of care;
a computing device configured to analyze the image of the at least one test strip captured by the sensor in order to determine the condition of the patient at the point of care;
a motor configured to position the at least one test strip near the sensor after the at least one test strip is exposed to the portion of the midstream urine sample; and
an additional collection component configured to collect an additional portion of the midstream urine sample from the initial collection component for central laboratory testing; and
a toilet bowl onto which the device is attached.

20. A method comprising:
collecting a midstream urine sample from a patient in an initial collection component;
transporting a portion of the midstream urine sample from the initial collection component to at least one test strip using a fluid transportation system;
exposing, by the fluid transportation system, the at least one test strip to the portion of the midstream urine sample, wherein the at least one test strip is one of a plurality of test strips configured to indicate a condition of the patient when exposed to the midstream urine sample;
positioning, by a motor, the at least one test strip near a sensor;
capturing an image of the at least one test strip using the sensor, wherein the image indicates the condition of the patient at a point of care;
analyzing, by a computing device, the image of the at least one test strip in order to determine the condition of the patient at the point of care; and
collecting, from the initial collection component, an additional portion of the midstream urine sample in an additional collection component for central laboratory testing.
